(19) United States
(12) Patent Application Publication
(10) Pub. No.: US 2025/0207201 A1
(43) Pub. Date: Jun. 26, 2025

(54) SYSTEM, METHOD, AND COMPUTER READABLE MEDIUM FOR DIAGNOSING AN INDIVIDUAL'S PERSONAL SALT INDEX

(71) Applicant: University of Virginia Patent Foundation, Charlottesville, VA (US)

(72) Inventor: Robin A. Felder, Charlottesville, VA (US)

(21) Appl. No.: 18/990,804

(22) Filed: Dec. 20, 2024

Related U.S. Application Data
(60) Provisional application No. 63/612,707, filed on Dec. 20, 2023.

(51) Int. Cl. C12Q 1/6883

(52) U.S. Cl. CPC C12Q 1/6883; C12Q 2600/106; C12Q 2600/156; C12Q 2600/158; C12Q 2600/178

(57) ABSTRACT

Described herein are systems, methods, and computer readable medium for, among other things, determining an individual's personal salt index. Such systems, methods, and computer readable medium can be utilized for providing information that can help guide healthcare providers by making informed decisions about treatments based on the individuals salt index. Such informed decisions can help mitigate the worsening or possibility of an adverse cardiovascular event in an individual whose salt index is unknown and receiving a treatment modality that may not be suitable for their particular genotype, phenotype, or any combination thereof. An aspect of the present disclosure also provides a system, method and computer readable medium for, among other things, algorithms/models that enabled the determination of a individual salt index to enable the therapeutic intervention. Also described herein are compositions, pharmaceutical compositions, kits, and methods of administration relating to therapeutics for blood pressure normalization and/or pathological cardiovascular diseases/phenotypes.

### CROSS REFERENCE TO RELATED APPLICATION[S]

This application claims priority to, and the benefit of, U.S. Provisional Patent Application Ser. No. 63/612,707 filed on Dec. 20, 2023, which is incorporated herein by reference in its entirety.

### STATEMENT REGARDING FEDERALLY SPONSORED RESEARCH OR DEVELOPMENT

This invention was made with government support under Grant No. HL074940 awarded by the National Institutes of Health. The Government has certain rights in the invention.

### BACKGROUND

Studies and clinical knowledge to date have demonstrated that there exists a wide variety of variability in the salt-sensitivity of blood pressure of individuals. Determination of the cause of the wide variability in BP with sodium intake continues to be investigated, but the variable response to sodium is complicated by the fact that BP is influenced by environment, genetics, epigenetics and behavior. Recent studies by the present inventors have shown that the distribution of the response to sodium intake, in humans is trinary (i.e. salt-resistant, SR, salt-sensitive, SS, and inverse salt-sensitive, ISS. While the trinary nature of the response has been demonstrated with remarkable robust statistics, making comparisons among studies is difficult since no consensus on salt dietary protocols or agreement about the MAP cutoff to use to define each group. Studies and clinical knowledge to date have also demonstrated that existing blood pressure medications may mask the fact that they are only treating the elevated blood pressure but not the effect of elevated salt on end organ damage. The previous binary classification of salt sensitivity ignored the third subset, namely inverse salt sensitive individuals who have an elevated blood pressure on a low salt diet. For example, the fact that a patient can be normotensive and still be classified as SS or ISS and will have significant cardiovascular disease further emphasizes the need for novel diagnostic paradigms beyond the blood pressure cuff. Accordingly, there is a need to address the aforementioned deficiencies and inadequacies in order to better address the BP response to salt intake in the human trinary population.

### SUMMARY

Described herein are systems, methods, computer readable media, and methods of treatment relating to determination of an individual's personal salt index.

In embodiments, described herein are methods of determining the salt sensitivity of a subject. Methods can comprise classifying a subject's blood pressure status as normotensive or hypertensive according to one or more first blood pressure measurements of the subject; and/or measuring the rate of sodium excretion from the human body over a period of time following a high and then low sodium diets in random order whereas excessively rapid sodium excretion from the body defining ISS and a slow excretion of sodium defining SS relative to the rate of sodium excretion in salt resistant individuals.

In embodiments, methods can comprise measuring the rate of sodium excretion from the body of the subject over a period of time following a high and then low sodium diets in random order, wherein excessive rapid sodium excretion from the body of the subject determines an inverse salt-sensitive status (ISS) for the subject, and a slow excretion of sodium determines salt-sensitive status relative to the rate of sodium excretion in one or more salt resistant (SR) individuals, or both.

In embodiments, methods can comprise determining the future hypertension, salt sensitivity (SS), or inverse salt-sensitivity (ISS) status of the subject by identifying, in a biological sample from the subject, the presence of: one or more sodium-bicarbonate cotransporter NBCe2 (SLC4a5) polymorphisms for SS status determination, one or more G protein-coupled receptor kinase 4 (GRK4) polymorphisms for hypertensive blood pressure status determination and SS status determination, one or more sodium channel epithelial 1 subunit alpha (αENaC) polymorphisms for ISS status determination, and one or more dopamine receptor D2 (DRD2) polymorphisms for ISS status determination.

Methods can comprise prescribing a low salt diet or high salt diet to the subject based on the subject's blood pressure status and determined future hypertension, salt sensitivity, or inverse salt-sensitivity status of the subject, or pharmacogenetic testing. In embodiments, a high salt diet can be prescribed to the normotensive subject having one or more SLC4a5polymorphisms, a low salt diet can be prescribed to the hypertensive subject having three or more GRK4 polymorphisms, a low salt diet can be prescribed to the hypertensive subject having one or more αENaC polymorphisms and one or more GRK4 polymorphisms, and a high salt diet can be prescribed to the hypertensive subject having one or more DRD2polymorphisms.

Methods can comprise classifying a subject's salt-sensitive blood pressure status as salt-sensitive, salt-resistance, or inverse-salt sensitive after following the prescribed low-salt or high-salt diet according to one or more second blood pressure measurements of the subject.

Methods an comprise identifying, from the one or more second blood pressure measurements of the subject, a salt-sensitivity status of the subject after following the prescribed low-salt or high-salt. The classifications can be salt-sensitive if mean arterial pressure (MAP) is controlled by a dietary salt reduction; salt-resistant if MAP unaffected by sodium; salt-resistant controlled by diet; salt-resistant essential hypertensive; inverse salt-sensitive controlled by diet; or salt-resistant with essential hypertension of the hypertensive subject not having a MAP not controlled by diet.

In embodiments, the subject is hypertensive if the one or more first blood pressure measurements are ≥130/80 mm Hg systolic/diastolic or is normotensive if the one or more first blood pressure measurements are <130/80 mm Hg systolic/diastolic.

In embodiments, the sample is a urine sample or spot urine sample. In embodiments, the sample comprises genomic DNA of the subject. In embodiments, the sample comprises retinal proximal tubule cells (RPTCs) of the subject.

In embodiments, the one or more SLC4A5 polymorphisms comprise rs7571842, rs1017783, or both.

In embodiments, the one or more GRK4 polymorphisms comprise R64L, A142V, A486V, or any combination of any thereof.

In embodiments, the one or more αENaC polymorphisms comprise at least rs4764586.

In embodiments, the one or more DRD2 polymorphisms comprise rs6276, rs6277, or both.

In embodiments, the high salt diet comprises high salt intake of about 300 mmol/day and the low salt diet comprises low salt intake of about 10 mm/day. In embodiments, the subject is on the high salt diet or low salt diet for at least 7 days.

In embodiments, the subject is salt-sensitive if the one or more first second pressure measurements demonstrate a >+5 mmHg change in MAP after the salt-diet, inverse salt-sensitive if the one or more first second pressure measurements demonstrate a ≥−5 mmHg change in MAP after the salt-diet, and salt-resistant if the subject exhibits <+5 mmHg change in MAP or <−5 mmHg change in MAP after the salt diet. In embodiments, the subject is salt-sensitive if the one or more first second pressure measurements demonstrate a ≥+7 mmHg change in MAP after the salt-diet, inverse salt-sensitive if the one or more first second pressure measurements demonstrate a >−7 mmHg change in MAP after the salt-diet, and salt-resistant if the subject exhibits <+7 mmHg change in MAP or <−7 mmHg change in MAP after the salt diet.

In embodiments, methods comprise, determining, from a sample of a normotensive subject not having one or more SLC4A5 polymorphisms a level of dopamine type 1 receptor (D₁R) expression, D₁R activity, or both; a level of Ang II type 2 receptor (AT₂R) expression, AT₂R activity, or both; determining the presence of one or more micro ribonucleic acids (miRNA) associated with salt-sensitivity or inverse salt-sensitivity, or any combination of any thereof. In embodiments, methods comprise, determining, from a sample of a normotensive subject or hypertensive subject a level of dopamine type 1 receptor (D₁R) expression, D₁R activity, or both; a level of Ang II type 2 receptor (AT₂R) expression, AT₂R activity, or both; determining the presence of one or more micro ribonucleic acids (miRNA) associated with salt-sensitivity or inverse salt-sensitivity, or any combination of any thereof. In embodiments, a level of dopamine type 1 receptor (D₁R) expression, D₁R activity, or both; a level of Ang II type 2 receptor (AT₂R) expression, AT₂R activity, or both; determining the presence of one or more micro ribonucleic acids (miRNA) associated with salt-sensitivity or inverse salt-sensitivity, or any combination of any thereof, are only assessed in normotensive subject not having one or more SLC4A5 polymorphisms.

In embodiments, methods as described herein comprise identifying the subject as salt-sensitive if the subject has: altered D₁R expression, activity, or both compared to a wild-type normotensive control, altered AT₂R expression, activity, or both compared to a wild-type normotensive control; or the presence of one or more micro ribonucleic acid (miRNA) associated with salt-sensitivity or inverse salt-sensitivity.

In embodiments, one or more miRNA associated with salt-sensitivity or inverse salt-sensitivity comprises at least miR-485-5p. In embodiments, methods as described herein further comprise prescribing a dietary salt reduction to the normotensive subject identified as salt-sensitive.

In embodiments, methods described herein further comprise prescribing, administering, or both: an anti-hypertensive pharmacogenetic test for anti-hypertensive therapeutics for the subject identified as salt-resistant with essential hypertension.

In embodiments, an anti-hypertensive pharmacogenetic test comprises determining the presence of one or more GRK4 polymorphisms from the identified one or more GRK4 polymorphisms of the biological sample or from a second biological sample from the subject. In embodiments, one or more GRK4 polymorphisms comprise R65L, A142V, A486V, individually or in any combination or any thereof.

Methods as described herein can further comprise prescribing, to the subject with a classified salt sensitivity or salt-resistant with essential hypertension, at least: a low salt diet, β-adrenergic blockers, or both, to the subject having wild-type GRK4 and the GRK4 polymorphisms R65L, A142V, and A486V; a non-β-adrenergic blocker to the subject having one or more wild-type GRK4 allelic variants and the GRK4 polymorphisms R65L and A142V; one or more β-adrenergic blockers, one or more AT₁R blockers, or any combination of any thereof to the subject having one or more wild-type GRK4 allelic variants and the GRK4 polymorphism A142V; a low-salt diet and diuretics to the subject having one or more wild-type GRK4 allelic variants and at least the GRK4 polymorphisms R65L, A142V, A486V; and one or more AT₁R blockers and one or more diuretics to the subject having one or more wild-type GRK4 allelic variants and at least the GRK4 polymorphisms A142V and A486V.

In embodiments, methods as described herein can comprise determining, in the biological sample of the subject or a second biological sample from the subject: the presence of one or more polymorphisms in solute carrier family 5 member 3 (SLC5A3), the presence of one or more polymorphisms in solute carrier family 5 member 11 (SLC5A11), or both. In embodiments, at least one of the one or more SLC5A11 polymorphisms comprise rs11074656.

In an embodiment of a method of determining the salt index of a subject described herein, the method can comprise: classifying a subject's blood pressure status as normotensive or hypertensive according to one or more first blood pressure measurements of the subject; determining the future hypertension, salt sensitivity, or inverse salt-sensitivity status of the subject by identifying, in a biological sample from the subject: the presence of: one or more SLC4a5 polymorphisms, one or more GRK4 polymorphisms, one or more αENaC polymorphisms, and one or more DRD2 polymorphisms; prescribing a low salt diet or high salt diet to the subject based on the subject's blood pressure status and determined future hypertension, salt sensitivity, or inverse salt-sensitivity status of the subject, or pharmacogenetic testing, wherein: a high salt diet is prescribed to the normotensive subject having one or more SLC4a5 polymorphisms, a low salt diet is prescribed to the hypertensive subject having three or more GRK4 polymorphisms, a low salt diet is prescribed to the hypertensive subject having one or more αENaC polymorphisms and one or more GRK4 polymorphisms, a high salt diet is prescribed to the hypertensive subject having one or more DRD2 polymorphisms; classifying a subject's salt-sensitive blood pressure statues as salt-sensitive, salt-resistance, or inverse-salt sensitive after following the prescribed salt diet according to one or more second blood pressure measurements of the subject, wherein the subject is: salt-sensitive if the one or more first second pressure measurements demonstrate a ≥+5 mmHg change in MAP after the salt-diet, inverse salt-sensitive if the one or more first second pressure measurements demonstrate a ≥−5 mmHg change in MAP after the salt-diet, and salt-resistant if the subject exhibits <+5 mmHg change in MAP or <−5 mmHg change in MAP after the salt diet; identifying, from the one or more second blood pressure measurements of the subject, a salt-sensitivity status of the subject after following the prescribed salt diet as being: salt-sensitive if mean arterial pressure (MAP) is controlled by a dietary salt reduction, salt-resistant if MAP unaffected by sodium, salt-resistant controlled by diet, salt-resistant essential hypertensive, inverse salt-sensitive controlled by diet; or salt-resistant with essential hypertension to the hypertensive subject not having a MAP not controlled by diet.

In an embodiment of a method of determining the salt index of a subject described herein, the method can comprise: classifying a subject's blood pressure status as normotensive or hypertensive according to one or more first blood pressure measurements of the subject; determining the future hypertension, salt sensitivity, or inverse salt-sensitivity status of the subject by identifying, in a biological sample from the subject: the presence of: one or more SLC4a5 polymorphisms, one or more GRK4 polymorphisms, one or more αENaC polymorphisms, and one or more DRD2 polymorphisms; prescribing a low salt diet or high salt diet to the subject based on the subject's blood pressure status and determined future hypertension, salt sensitivity, or inverse salt-sensitivity status of the subject, or pharmacogenetic testing, wherein: a high salt diet is prescribed to the normotensive subject having one or more SLC4a5 polymorphisms, a low salt diet is prescribed to the hypertensive subject having three or more GRK4 polymorphisms, a low salt diet is prescribed to the hypertensive subject having one or more αENaC polymorphisms and one or more GRK4 polymorphisms, a high salt diet is prescribed to the hypertensive subject having one or more DRD2 polymorphisms; classifying a subject's salt-sensitive blood pressure statues as salt-sensitive, salt-resistance, or inverse-salt sensitive after following the prescribed salt diet according to one or more second blood pressure measurements of the subject, wherein the subject is: salt-sensitive if the one or more first second pressure measurements demonstrate a ≥+5 mmHg change in MAP after the salt-diet, inverse salt-sensitive if the one or more first second pressure measurements demonstrate a ≥−5 mmHg change in MAP after the salt-diet, and salt-resistant if the subject exhibits <+5 mmHg change in MAP or <−5 mmHg change in MAP after the salt diet; identifying, from the one or more second blood pressure measurements of the subject, a salt-sensitivity status of the subject after following the prescribed salt diet as being: salt-sensitive if mean arterial pressure (MAP) is controlled by a dietary salt reduction, salt-resistant if MAP unaffected by sodium, salt-resistant controlled by diet, salt-resistant essential hypertensive, inverse salt-sensitive controlled by diet; or salt-resistant with essential hypertension to the hypertensive subject not having a MAP not controlled by diet; and prescribing, administering, or both, an anti-hypertensive pharmacogenetic test for anti-hypertensive therapeutics if the subject is identified as salt-resistant with essential hypertension, wherein the anti-hypertensive pharmacogenetic test comprises determining the presence of one or more GRK4 polymorphisms from the identified one or more GRK4 polymorphisms of the biological sample or from a second biological sample from the subject; and prescribing, to the subject with a classified salt sensitivity of salt-resistant with essential hypertension, at least: a low salt diet, β-adrenergic blockers, or both, to the subject having wild-type GRK4 and the GRK4 polymorphisms R65L, A142V, and A486V; a non-β-adrenergic blocker to the subject having one or more wild-type GRK4 allelic variants and the GRK4 polymorphisms R65L and A142V; one or more β-adrenergic blockers, one or more AT₁R blockers, or any combination of any thereof to the subject having one or more wild-type GRK4 allelic variants and the GRK4 polymorphism A142V; a low-salt diet and diuretics to the subject having one or more wild-type GRK4 allelic variants and at least the GRK4 polymorphisms R65L, A142V, A486V; and one or more AT₁R blockers and one or more diuretics to the subject having one or more wild-type GRK4 allelic variants and at least the GRK4 polymorphisms A142V and A486V.

In embodiments, the one or more GRK4 polymorphisms comprise R65L, A142V, A486V, individually or in any combination or any thereof.

In embodiments, methods as described herein can comprise determining, in the biological sample of the subject or a second biological sample from the subject: the presence of one or more polymorphisms in SLC5A3, the presence of one or more polymorphisms in SLC5A11, or both. In embodiments, the least one of the one or more SLC5A11 polymorphisms comprise rs11074656.

Methods as described herein can further comprise providing or otherwise collecting a biological sample from a subject. In embodiments, the biological sample of the subject can be a urine or spot urine sample. Methods as described herein can further comprise taking one or more blood measurements of a subject (i.e., first BP measurements, second BP measurements, or both). Methods as described herein can further comprise generating a report containing any aspect of identification, classification, or phenotype of the subject, for example, normotensive vs. hypertensive, SS vs. SR vs. ISS, and the like. Methods as described herein can further comprise

Described herein as systems containing hardware that can be configured to execute any method step or combination thereof. Described herein are computer-readable media that can contain instructions, that when executed by a computing device, perform any one or more of the method steps in any one or more combinations.

Described herein are kits. In embodiments, kits as described herein can comprise one or more primers or probes for determining, in a biological sample from a subject, the presence of: one or more SLC4a5 polymorphisms, one or more GRK4 polymorphisms, one or more αENaC polymorphisms, one or more DRD2 polymorphisms, or any combination of any thereof; and instructions for use, wherein the instructions for use comprise instructions for the user of the kit to perform at least any method as described herein.

In embodiments, kits further a sample collection device. In embodiments, instructions for use further comprise instructions for using the sample collection device. In embodiments, the sample collection device is a urine or spot urine collection device. In embodiments, the instructions are printed instructions on paper or plastic, or electronic instructions in the form of a electronic document, such as, a webpage, HTML, PDF, Word document, and the like.

In embodiments, kits further comprise one or more primers or probes for determining, in a biological sample from a subject, the presence of one or more polymorphisms in SLC5A3, the presence of one or more polymorphisms in SLC5A11, or both. In embodiments, the one or more SLC4A5 polymorphisms comprise rs7571842, rs1017783, or both. In embodiments, the one or more GRK4 polymorphisms comprise R64L, A142V, A486V, and any combination of any thereof. In embodiments, the one or more αENaC polymorphisms comprise rs4764586. In embodiments, the one or more DRD2 polymorphisms comprise rs6276, rs6277, or both. In embodiments, at least one of the one or more SLC5A11 polymorphisms comprise rs11074656.

In embodiments, kits further comprise one or more of: one or more primers or probes for determining the level of D₁R expression, one or more primers or probes for determining the level of AT₂R expression, one or more primers or probes for determining the presence of one or more micro ribonucleic acid (miRNA) associated with salt-sensitivity or inverse salt-sensitivity, or any combination of any thereof. In embodiments, one or more miRNA associated with salt-sensitivity or inverse salt-sensitivity comprises at least miR-485-5p.

### BRIEF DESCRIPTION OF THE DRAWINGS

Many aspects of the disclosed devices and methods can be better understood with reference to the following drawings. The components in the drawings are not necessarily to scale, emphasis instead being placed upon clearly illustrating the relevant principles. Moreover, in the drawings, like reference numerals designate corresponding parts throughout the several views.

FIG. 1: Each spot in this graph represents an individual's mean arterial blood pressure (MAP) response to a change in dietary sodium between 10 mmol and 300 mmol, each lasting a week. We ranked each individual's MAP according to the directional change in mm Hg grouping ISS individuals on the left and SS individuals on the right. The study participants were randomized as to whether they started with the high sodium diet for one week or the low sodium diet for one week to avoid directional bias. The cutoff between the SS individuals and the SR individuals was a positive 7 mm Hg or greater. Notice that the distribution of SR responses is linear and thus Gaussian. The distributions of SS and ISS MAP responses are non-linear and significantly different from the SR individuals. The ISS individuals have a reduction in MAP when consuming the 300 mmol sodium diet and an increase in MAP when consuming the 10 mmol sodium diet. Using K-Means clustering method (Squared Euclidean Distance), the data was categorized into three clusters in 3 different colors.

FIG. 2 is an embodiment of a decision tree for classifying a patient as being either salt resistant (SR), salt sensitive (SS), inverse salt sensitive (ISS), or having essential hypertension that can't be treated by adjusting dietary salt. The bottom three rows of decision boxes may assist a physician or other healthcare provider in the most likely anti-hypertensive medications to prescribe. A detailed description of each step in the decision tree is in the review's section entitled “Clinical Implications.”

FIG. 3 illustrates a putative mechanism behind the etiology of inverse salt sensitivity is an increased renal renin angiotensin system due to, in part, variants in the dopamine type 2 receptor (D₂R) which normally keeps the renin angiotensin system in check by inhibiting the angiotensin type 1 receptor (AT₁R). The increased sodium reabsorption stimulated by the overactive AT₁R on a low sodium diet raises MAP. An inverse salt sensitive individual on high sodium experiences a reduced MAP due to an overactive AT₂R which is natriuretic thus lowering MAP. Other SNPs are likely to be involved, leading to the hypothesis that an individual's personal salt index is determined by the sum total of increased and decreased sodium reabsorption in the kidney.

FIG. 4 is a is a block diagram illustrating an example of a machine upon which one or more aspects of embodiments of the present disclosure can be implemented.

### DETAILED DESCRIPTION

Before the present disclosure is described in greater detail, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Although example embodiments of the present disclosure are explained in some instances in detail herein, it is to be understood that other embodiments are contemplated. Accordingly, it is not intended that the present disclosure be limited in its scope to the details of construction and arrangement of components set forth in the following description or illustrated in the drawings. The present disclosure is capable of other embodiments and of being practiced or carried out in various ways.

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Regarding machine hardware, it should be appreciated that any of the components or modules referred to with regards to any of the present invention embodiments discussed herein, may be integrally or separately formed with one another. Further, redundant functions or structures of the components or modules may be implemented. Moreover, the various be communicated locally and/or remotely with any components may user/operator/customer/client or machine/system/computer/processor. Moreover, the various components may be in communication via wireless and/or hardwire or other desirable and available communication means, systems and hardware. Moreover, various components and modules may be substituted with other modules or components that provide similar functions.

Embodiments of the present disclosure will employ, unless otherwise indicated, techniques of genetics, biochemistry, molecular biology, cellular biology, cytology, tissue culture, clinical sample collection, therapeutic administrations, and the like.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only and is not intended to be limiting. It is also possible in the present disclosure that steps can be executed in different sequence where this is logically possible.

### Definitions

Unless otherwise defined, all technical and scientific terms used herein have the same meaning as commonly understood by one of ordinary skill in the art. Although methods and materials similar or equivalent to those described herein can be used in the practice or testing of the present disclosure, suitable methods and materials are described herein.

As used in the specification and the appended claims, the singular forms “a,” “an,” and “the” may include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently disclosed subject-matter.

The term “about”, when used herein in reference to a value, refers to a value that is similar, in context to the referenced value. In general, those skilled in the art, familiar with the context, will appreciate the relevant degree of variance encompassed by “about” in that context, for example, ±5%, ±4%, ±3%, ±2%, etc.

Two events or entities are “associated” with one another, as that term is used herein, if the presence, level and/or form of one is correlated with that of the other. For example, a particular entity (e.g., polypeptide, genetic signature, metabolite, microbe, etc.) is considered to be associated with a particular disease, disorder, or condition, if its presence, level and/or form correlates with incidence of and/or susceptibility to the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

As used herein, the term “comparable” refers to two or more agents, entities, situations, sets of conditions, etc., that may not be identical to one another but that are sufficiently similar to permit comparison there between so that one skilled in the art will appreciate that conclusions can reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc. to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Those skilled in the art will appreciate that the term “composition”, as used herein, can be used to refer to a discrete physical entity that comprises one or more specified components. In general, unless otherwise specified, a composition can be of any suitable form—e.g., gel, liquid, solid, etc.

A composition or method described herein as “comprising” one or more named elements or steps is open-ended, meaning that the named elements or steps are essential to a particular aspect or embodiment, but other elements or steps can be added within the scope of the composition or method. To avoid prolixity, it is also understood that any composition or method described as “comprising” (or which “comprises”) one or more named elements or steps also describes the corresponding, more limited composition or method “consisting essentially of” (or which “consists essentially of”) the same named elements or steps, meaning that the composition or method includes the named essential elements or steps and can also include additional elements or steps that do not materially affect the basic and novel characteristic(s) of the composition or method. It is also understood that any composition or method described herein as “comprising” or “consisting essentially of” one or more named elements or steps also describes the corresponding, more limited, and closed-ended composition or method “consisting of” (or “consists of”) the named elements or steps to the exclusion of any other unnamed element or step. In any composition or method disclosed herein, known or disclosed equivalents of any named essential element or step can be substituted for that element or step.

In this disclosure, “consisting essentially of” or “consists essentially” or the like, when applied to methods and compositions encompassed by the present disclosure refers to compositions like those disclosed herein, but which may contain additional steps, genotypes, phenotypes, structural groups, composition components or method steps (or analogs or derivatives thereof as discussed above). Such additional structural groups, composition components or method steps, etc., however, do not materially affect the basic and novel characteristic(s) of the compositions or methods, compared to those of the corresponding compositions or methods disclosed herein. “Consisting essentially of” or “consists essentially” or the like, when applied to methods and compositions encompassed by the present disclosure have the meaning ascribed in U.S. Patent law and the term is open-ended, allowing for the presence of more than that which is recited so long as basic or novel characteristics of that which is recited is not changed by the presence of more than that which is recited, but excludes prior art embodiments.

As used herein, “Improved,” “increased” or “reduced,” or grammatically comparable comparative terms, indicate values that are relative to a baseline value or reference measurement. For example, in some embodiments, an assessed value achieved with an agent of interest may be “improved” relative to that obtained or expected in the absence of treatment or with a comparable reference agent or control. Alternatively, or additionally, in some embodiments, an assessed value achieved with an agent of interest may be “improved” relative to that obtained in the same subject or system under different conditions (e.g., prior to or after an event such as administration of an agent of interest), or in a different, comparable subject (e.g., in a comparable subject or system that differs from the subject or system of interest). In some embodiments, comparative terms refer to statistically relevant differences (e.g., that are of a prevalence and/or magnitude sufficient to achieve statistical relevance). Those skilled in the art will be aware, or will readily be able to determine, in a given context, a degree and/or prevalence of difference that is required or sufficient to achieve such statistical significance.

As used herein, “isolated” means separated from constituents that otherwise may be present, for example, separated from bacterial stains or species that are not desired, or separating from other constituents that may be present with the micro-organisms in nature.

As used herein, the term “encode” refers to principle that DNA can be transcribed into RNA, which can then be translated into amino acid sequences that can form proteins

As used herein, the terms “optional” or “optionally” means that the subsequently described event or circumstance can or cannot occur, and that the description includes instances where said event or circumstance occurs and instances where it does not.

As used herein, “individual”, “organism”, “host”, “subject”, and “patient” refers to any living entity comprised of at least one cell. A living organism can be as simple as, for example, a single isolated eukaryotic cell or cultured cell or cell line, or as complex as a mammal, including a human being, and animals (e.g., vertebrates, amphibians, fish, mammals, e.g., cats, dogs, horses, pigs, cows, sheep, rodents, rabbits, squirrels, bears, primates (e.g., chimpanzees, gorillas, and humans). These terms (“individual,” “subject,” “host,” and “patient”) used interchangeably herein also refer to any mammalian subject for whom diagnosis, treatment, or therapy is desired, particularly humans. In embodiments, subject may relate to particular components of the subject, for instance specific tissues or fluids of a subject (e.g., human tissue in a particular area of the body of a living subject), which may be in a particular location of the subject, referred to herein as an “area of interest” or a “region of interest.” In embodiments, the region of interest is the blood and corresponding blood pressure, and the kidneys (in any part thereof or in whole). In embodiments, methods as described herein are only administered to humans.

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents and are meant to include future updates.

Reference throughout this specification to “one embodiment”, “an embodiment”, “another embodiment”, “some embodiment,” means that a particular feature, structure or characteristic described in connection with the embodiment is included in at least one embodiment of the present invention. Thus, appearances of the phrases “in one embodiment,” “in an embodiment,” “in another embodiment”, or “in some embodiment” in various places throughout this specification are not necessarily all referring to the same embodiment, but they may. Furthermore, the particular features, structures or characteristics may be combined in any suitable manner, as would be apparent to a person skilled in the art from this disclosure, in one or more embodiments. Furthermore, while some embodiments described herein include some, but not other, features included in other embodiments, combinations of features of different embodiments are meant to be within the scope of the invention. For example, in the appended claims, any of the claimed embodiments can be used in any combination.

A “control” sample or value refers to a sample that serves as a reference, usually a known reference, for comparison to a test sample or condition. For example, a test sample can include cells exposed to a test condition or a test agent, while the control is not exposed to the test condition or agent (e.g., negative control). The control can also be a positive control, e.g., a known primary cell or a cell exposed to known conditions or agents, for the sake of comparison to the test condition. A control can also represent an average value gathered from a plurality of samples, e.g., to obtain an average value. For therapeutic applications, a sample obtained from a patient suspected of having a given disorder or deficiency can be compared to samples from a known normal (non-deficient) individual. A control can also represent an average value gathered from a population of similar individuals, e.g., patient having a given deficiency or healthy individuals with a similar medical background, same age, weight, etc. A control value can also be obtained from the same individual, e.g., from an earlier-obtained sample, prior to the disorder or deficiency, or prior to treatment. One of skill will recognize that controls can be designed for assessment of any number of parameters.

The term “biological sample” encompasses a variety of sample types obtained from an organism or a cell line. The term encompasses blood and other liquid samples of biological origin, solid tissue samples, such as a biopsy specimen or tissue cultures or cells derived therefrom and the progeny thereof. The term includes samples that have been manipulated in any way after their procurement, such as by treatment with reagents, solubilization, or enrichment for certain components. The term includes a clinical sample, and includes cells in cell culture, cell supernatants, cell lysates, serum, plasma, biological fluids, and tissue samples.

The term “clinical well-being” as used herein, refers to a state or degree of clinical or physiological wellness or health of a patient. A clinician can evaluate a patient's clinical well-being by physical examination or performing one or more tests or assays.

“Inhibitors,” “activators,” and “modulators” of expression or of activity are used to refer to inhibitory, activating, or modulating molecules, respectively, identified using in vitro and in vivo assays for expression or activity of a described target protein (or encoding polynucleotide), e.g., ligands, agonists, antagonists, and their homologs and mimetics. The term “modulator” includes inhibitors and activators. Inhibitors are agents that, e.g., inhibit expression or bind to, partially or totally block stimulation or protease inhibitor activity, decrease, prevent, delay activation, inactivate, desensitize, or down regulate the activity of the described target protein, e.g., antagonists. Activators are agents that, e.g., induce or activate the expression of a described target protein or bind to, stimulate, increase, open, activate, facilitate, enhance activation or protease inhibitor activity, sensitize or up regulate the activity of described target protein (or encoding polynucleotide), e.g., agonists. Modulators include naturally occurring and synthetic ligands, antagonists and agonists (e.g., small chemical molecules, antibodies and the like that function as either agonists or antagonists). Such assays for inhibitors and activators include, e.g., applying putative modulator compounds to cells expressing the described target protein and then determining the functional effects on the described target protein activity, as described above. Samples or assays comprising described target protein that are treated with a potential activator, inhibitor, or modulator are compared to control samples without the inhibitor, activator, or modulator to examine the extent of effect. Control samples (untreated with modulators) are assigned a relative activity value of 100%. Inhibition of a described target protein is achieved when the activity value relative to the control is about 80%, optionally 50% or 25, 10%, 5% or 1%. Activation of the described target protein is achieved when the activity value relative to the control is 110%, optionally 150%, optionally 200, 300%, 400%, 500%, or 1000-3000% or more higher.

The terms “administering,” “delivering,” and “introducing,” can be used interchangeably to indicate the introduction of a therapeutic composition or agent (e.g., compositions comprising one or more therapeutics as described herein) into the body of a subject. The therapeutic composition or agent can be administered through any appropriate means that results in the delivery of at least a portion of the composition or agent to a desired location in the subject such that the composition or agent retains its therapeutic capability. Useful methods of delivering the therapeutic include, but are not limited to, intravenous delivery, subcutaneous delivery, intradermal delivery, intracoronary delivery, intracardiac delivery, oral delivery, or any combination thereof.

The term “administered continuously” refers to the continuous delivery of a therapeutic agent, e.g., compound, molecule, peptide, biologic, chemical, etc. over a 24-hour period.

The term “therapeutically effective amount” refers to an amount of therapeutic agent effective to treat at least one symptom of a disease or disorder in a subject. In other words, such an amount is sufficient to bring about a beneficial or desired clinical effect. The “therapeutically effective amount” of the agent for administration may vary based upon the desired activity, the diseased state of the subject being treated, the dosage form, method of administration, subject factors such as the subject's sex, genotype, weight and age, the underlying causes of the condition or disease to be treated, the route of administration and bioavailability, the persistence of the administered agent in the body, evidence of natriuresis and/or diuresis, the type of formulation, and the potency of the agent.

As used herein, the terms “pharmaceutically acceptable” or “pharmacologically acceptable” refer to compositions that do not substantially produce adverse reactions, e.g., toxic, allergic, or immunological reactions, when administered to a subject.

The terms “therapy,” “treatment,” and “amelioration” refer to any reduction in the severity of symptoms, e.g., of a neurodegenerative disorder or neuronal injury. As used herein, the terms “treat” and “prevent” are not intended to be absolute terms. Treatment can refer to any delay in onset, amelioration of symptoms, improvement in patient survival, improved cognitive function or coordination, increase in survival time or rate, etc. The effect of treatment can be compared to an individual or pool of individuals not receiving the treatment, or to the same patient prior to treatment or at a different time during treatment. In some aspects, the severity of disease is reduced by at least 5% or 10%, as compared, e.g., to the individual before administration or to a control individual not undergoing treatment. In some aspects the severity of disease is reduced by at least 25%, 50%, 75%, 80%, or 90%, or in some cases, no longer detectable using standard diagnostic techniques. The terms “treating” or “treatment” as used herein refers to an alleviation of symptoms associated with a disorder or disease, or inhibition of further progression or worsening of those symptoms, or prevention or prophylaxis of the disease or disorder, or curing the disease or disorder. Similarly, as used herein, an “effective amount” or a “therapeutically effective amount” of a compound of the invention refers to an amount of the compound that alleviates, in whole or in part, symptoms associated with the disorder or condition, or halts or slows further progression or worsening of those symptoms or prevents or provides prophylaxis for the disorder or condition.

As used throughout, the terms “nucleic acid,” “nucleic acid sequence,” “oligonucleotide,” “nucleotides,” or other grammatical equivalents as used herein mean at least two nucleotides, either deoxyribonucleotides or ribonucleotides, or analogs thereof, covalently linked together. Polynucleotides are polymers of any length, including, e.g., 20, 50, 100, 200, 300, 500, 1000, 2000, 3000, 5000, 7000, 10,000, etc. A polynucleotide described herein generally contains phosphodiester bonds, although in some cases, nucleic acid analogs are included that may have at least one different linkage, e.g., phosphoramidate, phosphorothioate, phosphorodithioate, or O-methylphophoroamidite linkages, and peptide nucleic acid backbones and linkages. Mixtures of naturally occurring polynucleotides and analogs can be made; alternatively, mixtures of different polynucleotide analogs, and mixtures of naturally occurring polynucleotides and analogs may be made. The following are non-limiting examples of polynucleotides: a gene or gene fragment, exons, introns, messenger RNA (mRNA), transfer RNA, ribosomal RNA, ribozymes, cDNA, CRNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. The term also includes both double-and single-stranded molecules. Unless otherwise specified or required, the term polynucleotide encompasses both the double-stranded form and each of two complementary single-stranded forms known or predicted to make up the double-stranded form. A polynucleotide is composed of a specific sequence of four nucleotide bases: adenine (A), cytosine (C), guanine (G), thymine (T), and uracil (U) for thymine when the polynucleotide is RNA. Thus, the term “polynucleotide sequence” is the alphabetical representation of a polynucleotide molecule. Unless otherwise indicated, a particular polynucleotide sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues.

Unless otherwise indicated, a particular nucleic acid sequence also implicitly encompasses conservatively modified variants thereof, alleles, orthologs, SNPs, and complementary sequences as well as the sequence explicitly indicated.

As used herein, “cDNA” refers to a DNA sequence that is complementary to an RNA transcript in a cell. It is a man-made molecule. Typically, cDNA is made in vitro by an enzyme called reverse-transcriptase using RNA transcripts as templates.

As used herein with reference to the relationship between DNA, cDNA, CRNA, RNA, protein/peptides, and the like “corresponding to” or “encoding” (used interchangeably herein) refers to the underlying biological relationship between these different molecules. As such, one of skill in the art would understand that operatively “corresponding to” can direct them to determine the possible underlying and/or resulting sequences of other molecules given the sequence of any other molecule which has a similar biological relationship with these molecules. For example, from a DNA sequence an RNA sequence can be determined and from an RNA sequence a cDNA sequence can be determined.

As used herein, “gene” can refer to a hereditary unit corresponding to a sequence of DNA that occupies a specific location on a chromosome and that contains the genetic instruction for a characteristic(s) or trait(s) in an organism. The term gene can refer to translated and/or untranslated regions of a genome. “Gene” can refer to the specific sequence of DNA that is transcribed into an RNA transcript that can be translated into a polypeptide or be a catalytic RNA molecule, including but not limited to, tRNA, siRNA, piRNA, miRNA, long-non-coding RNA and shRNA. Gene products of genes discussed herein (mRNA, for example), are also contemplated by the present disclosure as assay targets for assays of the present disclosure

The word “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell (Sambrook et al., 1989 Molecular Cloning: A Laboratory Manual, 18.1-18.88).

The terms “polypeptide” and “peptide” are used interchangeably herein to refer to a polymer of amino acid residues in a single chain. The terms apply to amino acid polymers in which one or more amino acid residue is an artificial chemical mimetic of a corresponding naturally occurring amino acid, as well as to naturally occurring amino acid polymers and non-naturally occurring amino acid polymers. Amino acid polymers may comprise entirely L-amino acids, entirely D-amino acids, or a mixture of L-and D-amino acids. The term “protein” as used herein refers to either a polypeptide or a dimer (i.e., two) or multimer (i.e., three or more) of single chain polypeptides. The single chain polypeptides of a protein may be joined by a covalent bond, e.g., a disulfide bond, or non-covalent interactions. The terms “portion” and “fragment” are used interchangeably herein to refer to parts of a polypeptide, nucleic acid, or other molecular construct.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, y-carboxyglutamate, and O-phosphoserine. Amino acid analogs refer to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The amino acids in the polypeptides described herein can be any of the 20 naturally occurring amino acids, D-stereoisomers of the naturally occurring amino acids, unnatural amino acids and chemically modified amino acids. Unnatural amino acids (that is, those that are not naturally found in proteins) are also known in the art, as set forth in, for example, Zhang et al. “Protein engineering with unnatural amino acids,” Curr. Opin. Struct. Biol. 23 (4): 581-87 (2013); Xie et al. “Adding amino acids to the genetic repertoire,” Curr. Opin. Chem. Biol. 9 (6): 548-54 (2005); and all references cited therein. Beta and gamma amino acids are known in the art and are also contemplated herein as unnatural amino acids.

In accordance with standard nomenclature, amino acid residue sequences are denominated by either a three letter or a single letter code as indicated as follows, for example: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (lle, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V). “Protein” and “Polypeptide” can refer to a molecule composed of one or more chains of amino acids in a specific order. The term protein is used interchangeable with “polypeptide.” The order is determined by the base sequence of nucleotides in the gene coding for the protein. Proteins can be involved in the structure, function, and regulation of various functions.

The term “identity” or “substantial identity,” as used in the context of a polynucleotide or polypeptide sequence described herein, refers to a sequence that has at least 60% sequence identity to a reference sequence. Alternatively, percent identity can be any integer from 60% to 100%. Exemplary embodiments include at least: 60%, 65%, 70%, 75%, 80%, 85%, 88%, 90%, 91%, 92%, 93%, 94%, 95%, 96%, 97%, 98%, or 99%, as compared to a reference sequence using the programs described herein; preferably BLAST using standard parameters, as described below. One of skill will recognize that these values can be appropriately adjusted to determine corresponding identity of proteins encoded by two nucleotide sequences by taking into account codon degeneracy, amino acid similarity, reading frame positioning and the like.

For sequence comparison, typically one sequence acts as a reference sequence to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison may be conducted by the local homology algorithm of Smith & Waterman Add. APL. Math. 2:482 (1981), by the homology alignment algorithm of Needleman & Wunsch J. Mol. Biol. 48:443 (1970), by the search for similarity method of Pearson & Lipman Proc. Natl. Acad. Sci. (U.S.A.) 85:2444 (1988), by computerized implementations of these algorithms (e.g., BLAST), or by manual alignment and visual inspection.

Algorithms that are suitable for determining percent sequence identity and sequence similarity are the BLAST and BLAST 2.0 algorithms, which are described in Altschul et al. (1990) J. Mol. Biol. 215:403-10 and Altschul et al. (1977) Nucleic Acids Res. 25:3389-402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1977)). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.01, more preferably less than about 10⁻⁵, and most preferably less than about 10⁻²⁰.

The terms “co-administration” or “co-administered” as used herein refer to the administration of at least two compounds or agent(s) or therapies to a subject. In some embodiments, the co-administration of two or more agents/therapies is concurrent. In other embodiments, a first agent/therapy is administered prior to a second agent/therapy in this aspect, each component may be administered separately, but sufficiently close in time to provide the desired effect, in particular a beneficial, additive, or synergistic effect. Those of skill in the art understand that the formulations and/or routes of administration of the various agents/therapies used may vary. The appropriate dosage for co-administration can be readily determined by one skilled in the art. In some embodiments, when agents/therapies are co-administered, the respective agents/therapies are administered at lower dosages than appropriate for their administration alone. Thus, co-administration is especially desirable in embodiments where the co-administration of the agents/therapies lowers the requisite dosage of a known potentially harmful (e.g., toxic) agent(s).

The term “composition” as used herein refers to a product comprising the specified ingredients in the specified amounts, as well as any product which results, directly or indirectly, from combination of the specified ingredients in the specified amounts. Such a term in relation to a pharmaceutical composition is intended to encompass a product comprising the active ingredient(s), and the inert ingredient(s) that make up the carrier, as well as any product which results, directly or indirectly, from combination, complexation, or aggregation of any two or more of the ingredients, or from dissociation of one or more of the ingredients, or from other types of reactions or interactions of one or more of the ingredients. Accordingly, the pharmaceutical compositions of the present disclosure encompass any composition made by admixing a compound of the present disclosure and a pharmaceutically acceptable carrier.

When a compound of the present disclosure is used contemporaneously with one or more other drugs, a pharmaceutical composition containing such other drugs in addition to the compound of the present disclosure is contemplated. Accordingly, the pharmaceutical compositions of the present disclosure include those that also contain one or more other active ingredients, in addition to a compound of the present disclosure. The weight ratio of the compound of the present disclosure to the second active ingredient may be varied and will depend upon the effective dose of each ingredient. Generally, an effective dose of each will be used. Thus, for example, but not intended to be limiting, when a compound of the present disclosure is combined with another agent, the weight ratio of the compound of the present disclosure to the other agent will generally range from about 1000:1 to about 1:1000, preferably about 200:1 to about 1:200. Combinations of a compound of the present disclosure and other active ingredients will generally also be within the aforementioned range, but in each case, an effective dose of each active ingredient should be used. In such combinations the compound of the present disclosure and other active agents may be administered separately or in conjunction. In addition, the administration of one element may be prior to, concurrent to, or subsequent to the administration of other agent(s).

A composition of the disclosure can be a liquid solution, suspension, emulsion or a powder. Various delivery systems are known and can be used to administer a composition of the disclosure, e.g. encapsulation in liposomes, microparticles, microcapsules, and the like, and then delivered to a patient by means of such as a nebulizer.

Compositions for administration may include sterile aqueous or non-aqueous solvents, such as water, isotonic saline, isotonic glucose solution, buffer solution, or other solvents conveniently used for parenteral administration of therapeutically active agents, stabilizers, buffers, or preservatives, e.g. antioxidants such as methylhydroxybenzoate or similar additives.

A composition of the disclosure may be sterilized by, for example, addition of sterilizing agents to the composition, irradiation of the composition, or heating the composition. Alternatively, the compounds or compositions of the present disclosure may be provided as sterile solid preparations e.g. lyophilized powder, which are readily dissolved in sterile solvent immediately prior to use.

After pharmaceutical compositions have been prepared, they can be placed in an appropriate container and labeled for treatment of an indicated condition. For administration of a composition of the disclosure, such labeling would include amount, frequency, and method of administration.

The term “freeze-dried (lyophilized) as used herein refers to a preparation of nucleic acids or other aspects of the present disclosure that have been initially frozen and the water content removed by vacuum.

The term “pharmaceutically acceptable carrier” as used herein refers to a diluent, adjuvant, excipient, or vehicle with which a probe of the disclosure is administered and which is approved by a regulatory agency of the Federal or a state government or listed in the U.S. Pharmacopeia or other generally recognized pharmacopeia for use in animals, and more particularly in humans. Such pharmaceutical carriers can be liquids, such as water and oils, including those of petroleum, animal, vegetable or synthetic origin, such as peanut oil, soybean oil, mineral oil, sesame oil and the like. The pharmaceutical carriers can be saline, gum acacia, gelatin, starch paste, talc, keratin, colloidal silica, urea, and the like. When administered to a patient, the probe and pharmaceutically acceptable carriers can be sterile. Water is a useful carrier when the probe is administered intravenously. Saline solutions and aqueous dextrose and glycerol solutions can also be employed as liquid carriers, particularly for injectable solutions. Suitable pharmaceutical carriers also include excipients such as glucose, lactose, sucrose, glycerol monostearate, sodium chloride, glycerol, propylene, glycol, water, ethanol and the like. The present compositions, if desired, can also contain minor amounts of wetting or emulsifying agents, or pH buffering agents. The present compositions advantageously may take the form of solutions, emulsion, sustained-release formulations, or any other form suitable for use.

The term “preventing” means to stop or hinder a disease, disorder, or symptom of a disease or condition through some action.

The term “reducing” means to diminish in extent, amount, or degree.

The term “therapeutic agent” as used herein refers to a therapeutic substance selected from a group consisting of, but not limited to, analgesics, anesthetics, anti-inflammatory agents, antiasthma agents, antibiotics (including penicillins), anticoagulants, antihistamines, antitussives, antihypertensive agents, antimuscarinic agents, antimycobacterial agents, antioxidant agents, antipyretics, immunosuppressants, immunostimulants, antiviral agents, bacteriostatic agents, bronchodilators, buffering agents, contrast media, corticosteroids, cough suppressants (expectorants and mucolytics), diagnostic agents, diagnostic imaging agents, free radical scavenging agents, growth factors, haemostatics, immunological agents, lipid regulating agents, muscle relaxants, proteins, peptides and polypeptides, prostaglandins, radio-pharmaceuticals, time release binders, anti-allergic agents, stimulants and anoretics, steroids, sympathomimetics, vasodilators, and xanthines.

### Discussion

Described herein are systems, methods, and computer readable medium for, among other things, determining an individual's personal salt index. Such systems, methods, and computer readable medium can be utilized for providing information that can help guide healthcare providers by making informed decisions about treatments based on the individuals salt index. Such informed decisions can help mitigate the worsening or possibility of an adverse cardiovascular event in an individual whose salt index is unknown and receiving a treatment modality that may not be suitable for their particular genotype, phenotype, or any combination thereof. An aspect of the present disclosure also provides a system, method and computer readable medium for, among other things, algorithms/models that enabled the determination of an individual salt index to enable the therapeutic intervention. Also described herein are compositions, pharmaceutical compositions, kits, and methods of administration relating to therapeutics for blood pressure normalization and/or pathological cardiovascular diseases/phenotypes. Also described herein are kits relating to determination of an individual's salt index.

### I. Methodologies for Salt Index Determination

Described herein are methods, systems, platforms, and machine-readable media relating to the determination of a salt index of a subject, for example, a normotensive or hypertensive human subject that may or may not have blood pressure changes in response to salt challenges.

### A. Methodologies

Described herein are methods of identifying an individual's personal salt index. Methods as described herein can comprise: identifying a subject as normotensive or hypertensive according to a measured or provided blood pressure measurement; identifying the presence of one or more polymorphisms of one or more genes of interest (genes of interest described in section (A) (ii) below for a future risk determination of hypertensive, salt-sensitive, or inverse salt-sensitive, for example (i.e., renin-angiotensin system, renal dopamine receptors, and renal pumps and/or transporters associated with sodium secretion, particularly in the proximal tubules (e.g . . . , membrane-bound ionic transporters such as sodium transporters or cotransporters)); administering a low salt or high salt diet following the risk determination; and assessing the salt sensitivity of the blood pressure of the subject following the salt diet to identify a salt-sensitivity status of the individual.

Methods as described herein can further comprise additional pharmacogenetic testing, as well as the utilization of additional biochemical assays (expression and/or activity level determination) to determine a pharmacogenetic phenotype of the subject. Following the pharmacogenetic phenotype determination, the salt-sensitivity of blood pressure status can be identified, and an appropriate treatment regime can be prescribed and/or administered.

i. Blood Pressure Measurements

Methodologies as described herein can, in embodiments, begin with a measured blood pressure of a subject to determine a blood pressure status as normotensive or hypertensive. Normal blood pressure as defined as a BP less than about 130/80 mm Hg systolic/diastolic. Normotensive individuals with this blood pressure may still be salt sensitive in that their pressure increases greater than about 5 mm or about 7 mm Hg on a high salt diet. In embodiments, their pressure increases greater than about 5 mm Hg on a high salt diet. their pressure increases greater than about about 7 mm Hg on a high salt diet.

ii. Polymorphisms, Differential Activity, and miRNA Presence Associated with Personal Salt Indexes

Methods according to the present disclosure can utilize polymorphism identification of several key genes associated with, in particular, the renin-angiotensin system, renal dopamine receptors, and renal pumps and/or transporters associated with sodium secretion, particularly in the proximal tubules (i.e., membrane-bound ionic transporters such as sodium transporters).

Methods as described herein can comprise determining the presence of one or more polymorphisms in genes associated with sodium secretion from the kidney, for example, transporters and/or pumps in the proximal tubules (e.g., solute carrier family 4 member 5 SLC4A5).

Methods as described herein can comprise determining the presence of one or more polymorphisms in genes associated with the renin-angiotensin system (RAS), for example, G protein-coupled receptor kinase 4 (GRK4) and Epithelial Sodium Channel alpha subunit (αENaC).

Methods as described herein can comprise determining the presence of one or more polymorphisms in renal dopamine receptors, for example, dopamine receptor D2 (DRD2). It would be understood by the skilled artisan that ribonucleic acid gene products of genes above can also be analyzed for differential expression (mRNA levels, for example). Activity of protein gene products (i.e., proteins fragments and functional proteins encoded by genes described above) can also be analyzed, for example, with in vitro cellular assays (using an individuals RPTCs, for example). Details of such cellular assays, and primer sequences, can be found in the art and in Example 2, for example.

The presence of other nucleic acids, miRNA, for example, can also be examined and utilized for determination of an individual's personal salt index accordingly.

a. SLC4A5

Methods as described herein can comprise determining the presence of one or more polymorphisms in solute carrier family 4 member 5 (SLC4A5) (also known as NBC4; NBCe2; for example, NCBI Gene ID No: 57835). In embodiments, the one or more SLC4A5 polymorphisms can be rs7571842 (https://www.ncbi.nlm.nih.gov/snp/rs7571842, accessed Dec. 20, 2024, the entire contents of which are incorporated by reference as if fully set forth herein), rs10177833 (https://www.ncbi.nlm.nih.gov/snp/rs10177833, accessed Dec. 20, 2024, the entire contents of which are incorporated by reference as if fully set forth herein), or both.

Individuals who have SNPs in the sodium bicarbonate co-transporter (expressed in the proximal tubule of the kidney) (SLC4A5, rs7571842, rs10177833) have a higher risk for salt sensitivity and could benefit from a dietary reduction in salt. Individuals who do not express these SNPs are considered normotensive salt resistant individuals.

According to the present disclosure, SLC4A5 variants are strongly associated with salt sensitivity. In one aspect, the salt sensitivity and/or SLC4A5 determination is in a Caucasian subject.

SLC4A5 (Homo sapiens solute carrier family 4, sodium-bicarbonate co-transporter) has NCBI Reference Sequence Number NG_032663.1 and is a 134, 166 bp DNA. There are 2095 SNPs in SLC4A5. SNPs rs7571842 and rs10177833 are both located in the intron region having Ref. Seq. No. NM_021196.3.

rs7571842 has a single nucleotide variation and the reference/SNP alleles are A/G, the ancestral allele being G. The position on chromosome 2 is 74460904 (+).

rs 10177833 has a single nucleotide variation and the reference/SNP allele is A/C, the ancestral allele being C. The position on chromosome 2 is 74457718 (+).

With regard to identifying and measuring polymorphisms of a gene associated with salt sensitivity herein for diagnostic and monitoring purposes, the present disclosure further provides for measuring and comparing SLC4A5 expression, levels, and activity in subjects with varying genotypes to establish an individual's salt index, to establish treatment regimens based on the diagnostic assays measuring the polymorphisms and/or SLC4A5 expression levels, and activity which help determine which subjects are amenable to particular treatment regimens, as well as to monitor the progression of the treatment.

The skilled artisan would recognize and readily understand that primers and probes for determination of SLC4A5 variants are readily available in the art. Examples include, but are not limited to, TaqMan® assay ID C 197439_10 (for rs7571842, Thermo Fisher Scientific) and C 1137534_10 (for rs10177833, Thermo Fisher Scientific).

b. GRK4

Methods as described herein can comprise determining the presence of one or more polymorphisms in G protein-coupled receptor kinase 4 (GRK4) (also known as IT11; GPRK4; GRK4a; GPRK2L; for example, NCBI Gene ID No: 2868). GRK4 is located on chromosome 4 (4p16.3) and its NCBI Reference Seq. No. is NC—000004.11. The chromosome position of GRK4 SNP rs1801058 is 3039150. The amino acid position is 454. In embodiments, the one or more GRK4 polymorphisms can be one or more of R65L, A142V, and A486V. The one or more GRK4 polymorphisms can be present in any of the 6 total GRK4 alleles of the subject. The GRK4 polymorphisms above include nucleotide 448, CGT to CTT (amino acid 65R >L, rs2960306, https://www.ncbi.nlm.nih.gov/snp/rs7571842, accessed Dec. 20, 2024, the entire contents of which are incorporated by reference as if fully set forth herein); nucleotide 679,GCC to GTC (amino acid 142A >V, rs1024323, https://www.ncbi.nlm.nih.gov/snp/rs1024323, accessed Dec. 20, 2024, the entire contents of which are incorporated by reference as if fully set forth herein); and nucleotide 1711, GCG to GTG (amino acid 486A >V, rs1801058, https://www.ncbi.nlm.nih.gov/snp/rs1801058, accessed Dec. 20, 2024, the entire contents of which are incorporated by reference as if fully set forth herein).

Additional details regarding GRK4 structure and function, including the above-referenced polymorphisms, can be found, for example, in Yang J, Hall J E, Jose P A, Chen K, Zeng C. Comprehensive insights in GRK4 and hypertension: From mechanisms to potential therapeutics. Pharmacol Ther. 2022 November; 239:108194. doi: 10.1016/j.pharmthera.2022.108194. Epub 2022 Apr. 27. PMID: 35487286; PMCID: PMC9728143, the contents of which are incorporated by reference in its entirety.

The skilled artisan would recognize and readily understand that primers and probes for determination of GRK4 variants are readily available in the art. Examples include, but are not limited to, TaqMan® assay ID C. 755465_10 (for R65L/rs7571842, Thermo Fisher Scientific), C __ 8281910_10 (for A142V, rs1024323, Thermo Fisher Scientific), and C_26934425_10 (A486V, rs1801058 Thermo Fisher Scientific).

### c. αENaC

Methods as described herein can comprise determining the presence of one or more polymorphisms in Epithelial Sodium Channel alpha subunit (αENaC) (also known as BESC2; ENaCa; SCNEA; SCNN1; LIDLS3; PHA1B1; ENaCalpha; for example, NCBI Gene ID No: Gene ID: 6337). In embodiments, the one or more αENaC polymorphisms can be rs4764586 (https://www.ncbi.nlm.nih.gov/snp/rs4764586, accessed Dec. 20, 2024, the entire contents of which are incorporated by reference as if fully set forth herein). The skilled artisan would recognize and readily understand that primers and probes for determination of αENaC variants are readily available in the art. Examples include, but are not limited to, TaqMan® assay ID C_25603866_10 (for rs4764586, Thermo Fisher Scientific).

d. DRD2

Methods as described herein can comprise determining the presence of one or more polymorphisms in dopamine receptor D2 (DRD2) also known as D2R; D2DR; for example, NCBI Gene ID No: Gene ID: 1813). The dopamine type 2 receptor gene is located on chromosome 11. The NCBI Reference Sequence No. is NG_008841.1 (72685 bp DNA). There are two mRNA variants (NM 000795.3 and NM_016574.3). In one aspect, a dopamine type 2 receptor of the SNP disclosure is SNP rs6276(https://www.ncbi.nlm.nih.gov/snp/rs6276, accessed Dec. 20, 2024, the entire contents of which are incorporated by reference as if fully set forth herein), rs6277(https://www.ncbi.nlm.nih.gov/snp/rs6277, accessed Dec. 20, 2024, the entire contents of which are incorporated by reference as if fully set forth herein), or both. The chromosome 11 position of the rs6276 SNP is 113281397 (−). The skilled artisan would recognize and readily understand that primers and probes for determination of DRD2 variants are readily available in the art. Examples include, but are not limited to, TaqMan® assay ID C _326648_20 (for rs6276, Thermo Fisher Scientific), C_11339240_10 (for rs6277, Thermo Fisher Scientific)

e. D₁R and AT₂R

The G protein-coupled receptors dopamine type 1 receptor (D₁R) and the AT₂R increase the excretion of sodium (natriuretic) while αENaC and RAS aid in the renal tubular reabsorption of sodium (antinatriuretic).³⁵⁻³⁷ The relative abundance and/or activity of these receptors and channels in RPTCs have been found to correlate with the change in BP following a change in sodium intake that can serve as diagnostic tests for ISS, SR and SS.^{13,19,38} Polymorphisms in these receptors and channels are also associated with the expression of the ISS and SS phenotypes.¹³ In certain aspects, mRNA expression can be analyzed with known primers and probes and PCR, activity can be measured, for example, by quantifying membrane recruitment following a salt challenge or salt removal, for example, using in vitro cellular immunostaining assays. Such assays can be done even in cells from the subject, for example, RPTC that may be present in the urine sample or an individual.

Additional details relating to the these receptors, their activities, and polymorphisms, can be found, for example, in: Cuka E, Simonini M, Lanzani C, Zagato L, Citterio L, Messaggio E, Faienza S, Brioni E, Hamlyn J M, Manunta P. Inverse salt sensitivity: an independent risk factor for cardiovascular damage in essential hypertension. J Hypertens. 2022;40:1504-1512.

doi: 10.1097/HJH.0000000000003174; Jose P A, Yang Z, Zeng C, Felder R A. The importance of the gastrorenal axis in the control of body sodium homeostasis. Exp Physiol. 2016; 101:465-470. doi: 10.1113/EP085286; and Mente A, O'Donnell M, Rangarajan S, McQueen M, Dagenais G, Wielgosz A, Lear S, Ah S T L, Wei L, Diaz R, et al. Urinary sodium excretion, blood pressure, cardiovascular disease, and mortality: a community-level prospective epidemiological cohort study. Lancet. 2018;392:496-506. doi: 10.1016/S0140-6736 (18) 31376-X, the entireties of which are incorporated by reference as if fully set forth herein regarding at least receptor details, activity testing, and polymorphisms.

f. SLC5A3 and SLC5A11

Solute carrier family 5 member 3 (SLC5A3; also known as SMIT1, for example NCBI Gene ID: 6526) is a sodium/myoinositol cotransporter that are expressed in the human kidney. SMIT2 (SLC5A11, SGLT6) is also a sodium/myoinositol cotransporter expressed in the human kidney. Methods as described herein can comprise detecting one or more polymorphisms in either of these genes, for example, the SLC5A11 polymorphism rs11074656 (https://www.ncbi.nlm.nih.gov/snp/rs11074656, accessed Dec. 20, 2024, the entire contents of which are incorporated by reference as if fully set forth herein).

The skilled artisan would recognize and readily understand that primers and probes for determination of SLC5A3 and SLC5A11 variants are readily available in the art. Examples include, but are not limited to, TaqMan® assay ID C_ 3173341_30 (for SLC5A11 variant rs11074656, Thermo Fisher Scientific)

g. miRNA

DNA regulators such as micro-RNAs (miRNAs) are beginning to serve as disease markers. The association of urinary micro-RNAs with an individual's BP response to dietary sodium intake has been reported. A recent study with a microarray containing 1898 probes was used to study the human urinary exosomal miRNome; of 194 miRNAs 45 miRNAs had significant associations with SS or ISS. A total of 25 miRNAs were found to be associated with ISS.

Although not differentially expressed, miR-485-5p is one of the 194 miRNAs found, which binds to the D_{R}D2 rs6276, reducing D₂R expression. In human RPT cells, miR-217 which mediates the protective effects of D₂R on fibrosis of human RPTCs is regulated by D_{R}D2; D_{R}D2 rs6276 reduces D₂R expression. Furthermore, a miRNA mimic that behaved like the endogenous mir-485-5p miRNA reduced the expression of D₂R, but only in the RPTCs from the urine of individuals with ISS. A miRNA blocker inhibited endogenous miRNAs, resulting in normalization of the expression of the D₂R. K

Additional details of miRNA as described herein can be found, for example, in hella H W, Bakhet M, Lichner Z, Romaschin A D, Jewett M A, Yousef G M. MicroRNAs in Kidney Disease: An Emerging Understanding. Am J Kidney Dis. 2012. doi: 10.1053/j.ajkd.2012.09.018, the entire contents of which are incorporated by reference as if fully set forth herein.

iii. Polymorphism Determination

In embodiments, the polymorphisms described above can be determined from a biological sample from the subject. In embodiments, the biological sample is a urine or a spot urine sample. In embodiments, the biological sample comprises genomic DNA of a subject. In embodiments, the sample comprises one or more renal proximal tubule cells (RPTCs). In embodiments, the sample is a urine sample comprising one or more RPTCs. In embodiments, the sample is a spot urine sample comprising one or more RPTCs. In embodiments, the sample comprises nucleic acids isolated from one or more RPTCs of a subject. In embodiments, the sample comprises nucleic acids isolated from one or more urine-derived RPTCs of a subject. The skilled artisan would readily recognize that other samples can be utilized containing alternative sources of genomic DNA other than those listed above that can be utilized to determine the polymorphism status of any one or more genes discussed herein.

Methods of polymorphism determination are known in the art. Additional examples regarding polymorphism determination methodologies can be found, for example and without intending to be limiting, in at least paragraphs [0265]-[0286] of United States Patent Publication No. US 2015/0133414A1, the contents of which are incorporated by reference as if fully set forth herein. Additional methodologies include the use of commercially-available platforms and methodologies for polymorphism, single-nucleotide polymorphism (SNP) detection in particular, for example, the use of the TaqMan® platform or other well known and validated methods used in the art to determine an individuals genotype for these polymorphisms of interest.

iv. Salt Diets

Salt diets according to the present disclosure can, for example, comprise about a 7 mmol to about an 8 mmol intake daily to about a 9 mmol intake daily to about a 10 mmol intake daily to about an 11 mmol intake daily to about a 12 mmol intake daily to about a 13 mmol intake daily for seven days for a low-salt diet, and about a 270 to about a 280 to about a 290 to about a 300 mmol intake daily to about a 310 mmol intake daily to about a 320 mmol daily intake for seven days for a high-salt diet. Following the administration/compliance with the salt diet, a second blood pressure measurement can be taken to determine if the subject's BP normalizes to normotensive after relief from the salt diet or remains hypertensive.

### B. Systems for Salt Index Determination

Described herein are systems for executing one of more or all of the method steps described herein. Systems can comprise, for example, a machine readable medium, that, when executed, contains logic that executes any one or more of the steps of the method.

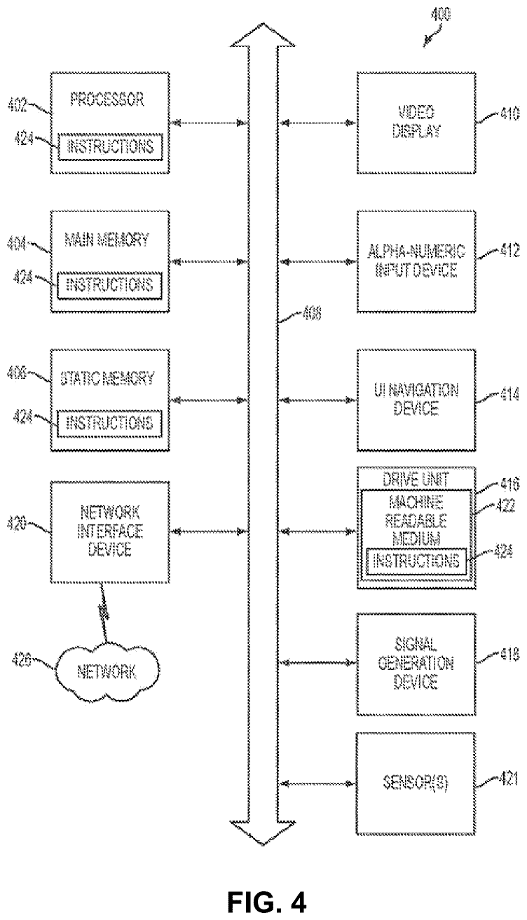

FIG. 4 is a block diagram illustrating an example of a machine upon which one or more aspects of embodiments of the present invention can be implemented (in particular, systems and machine-readable media that can perform the methods above).

Referring to FIG. 4, an aspect of an embodiment of the present invention includes, but not limited thereto, a system, method, and computer readable medium that provides for steps to determine a subject's salt index, which illustrates a block diagram of an example machine 400 upon which one or more embodiments (e.g., discussed methodologies) can be implemented (e.g., run).

Examples of a machine 400 can include logic, one or more components, circuits (e.g., modules), or mechanisms. Circuits are tangible entities configured to perform certain operations. In an example, circuits can be arranged (e.g., internally or with respect to external entities such as other circuits) in a specified manner. In an example, one or more computer systems (e.g., a standalone, client or server computer system) or one or more hardware processors (processors) can be configured by software (e.g., instructions, an application portion, or an application) as a circuit that operates to perform certain operations as described herein. In an example, the software can reside (1) on a non-transitory machine readable medium or (2) in a transmission signal. In an example, the software, when executed by the underlying hardware of the circuit, causes the circuit to perform the certain operations.

In an example, a circuit can be implemented mechanically or electronically. For example, a circuit can comprise dedicated circuitry or logic that is specifically configured to perform one or more techniques such as discussed above, such as including a special-purpose processor, a field programmable gate array (FPGA) or an application-specific integrated circuit (ASIC). In an example, a circuit can comprise programmable logic (e.g., circuitry, as encompassed within a general-purpose processor or other programmable processor) that can be temporarily configured (e.g., by software) to perform the certain operations. It will be appreciated that the decision to implement a circuit mechanically (e.g., in dedicated and permanently configured circuitry), or in temporarily configured circuitry (e.g., configured by software) can be driven by cost and time considerations.

Accordingly, the term “circuit” is understood to encompass a tangible entity, be that an entity that is physically constructed, permanently configured (e.g., hardwired), or temporarily (e.g., transitorily) configured (e.g., programmed) to operate in a specified manner or to perform specified operations. In an example, given a plurality of temporarily configured circuits, each of the circuits need not be configured or instantiated at any one instance in time. For example, where the circuits comprise a general-purpose processor configured via software, the general-purpose processor can be configured as respective different circuits at different times. Software can accordingly configure a processor, for example, to constitute a particular circuit at one instance of time and to constitute a different circuit at a different instance of time.

In an example, circuits can provide information to, and receive information from, other circuits. In this example, the circuits can be regarded as being communicatively coupled to one or more other circuits. Where multiple of such circuits exist contemporaneously, communications can be achieved through signal transmission (e.g., over appropriate circuits and buses) that connect the circuits. In embodiments in which multiple circuits are configured or instantiated at different times, communications between suet, circuits can be achieved, for example, through the storage and retrieval of information in memory structures to which the multiple circuits have access. For example, one circuit can perform an operation and store the output of that operation in a memory device to which it is communicatively coupled. A further circuit can then, at a later time, access the memory device to retrieve and process the stored output. In an example, circuits can be configured to initiate or receive communications with input or output devices and can operate on a resource (e.g., a collection of information).

The various operations of method examples described herein can be performed, at least partially, by one or more processors that are temporarily configured (e.g., by software) or permanently configured to perform the relevant operations. Whether temporarily or permanently configured, such processors can constitute processor-implemented circuits that operate to perform one or more operations or functions. In an example, the circuits referred to herein can comprise processor-implemented circuits.

Similarly, the methods described herein can be at least partially processor-implemented. For example, at least some of the operations of a method can be performed by one or processors or processor-implemented circuits. The performance of certain of the operations can be distributed among the one or more processors, not only residing within a single machine, but deployed across a number of machines. In an example, the processor or processors can be located in a single location (e.g., witt1in a t1ome environment, an office environment or as a server farm), while in other examples the processors can be distributed across a number of locations.

The one or more processors can also operate to support performance of the relevant operations in a “cloud computing” environment or as a “software as a service” (SaaS). For example, at least some of the operations can be performed by a group of computers (as examples of machines including processors), with these operations being accessible via a network (e.g., the Internet) and via one or more appropriate interfaces (e.g., Application Program Interfaces (APIs).)

Example embodiments (e.g., apparatus, systems, or methods) can be implemented in digital electronic circuitry, in computer hardware, in firmware, in software, or in any combination thereof. Example embodiments can be implemented using a computer program product (e.g., a computer program, tangibly embodied in an information carrier or in a machine readable medium, for execution by, or to control the operation of, data processing apparatus such as a programmable processor, a computer, or multiple computers).

A computer program can be written in any form of programming language, including compiled or interpreted languages, and it can be deployed in any form, including as a stand-alone program or as a software module, subroutine, or other unit suitable for use in a computing environment. A computer program can be deployed to be executed on one computer or on multiple computers at one site or distributed across multiple sites and interconnected by a communication network.

In an example, operations can be performed by one or more programmable processors executing a computer program to perform functions by operating on input data and generating output. Examples of method operations can also be performed by, and example apparatus can be implemented as, special purpose logic circuitry (e.g., a field programmable gate array (FPGA) or an application-specific integrated circuit (ASIC)).

The computing system can include clients and servers. A client and server are generally remote from each other and generally interact through a communication network.

The relationship of client and server arises by virtue of computer programs running on the respective computers and having a client-server relationship to each other. In embodiments deploying a programmable computing system, it will be appreciated that both hardware and software architectures require consideration. Specifically, it will be appreciated that tt1e choice of whether to implement certain functionality in permanently configured hardware (e.g., an ASIC), in temporarily configured hardware (e.g., a combination of software and a programmable processor), or a combination of permanently and temporarily configured hardware can be a design choice. Below are set out hardware (e.g., machine 400) and software architectures that can be deployed in example embodiments.

In an example, the machine 400 can operate as a standalone device or the machine 400 can be connected (e.g., networked) to other machines.

In a networked deployment, the machine 400 can operate in the capacity of either a server or a client machine in server-client network environments. In an example, machine 400 can act as a peer mact1ine in peer-to-peer (or other distributed) network environments. The machine 400 can be a personal computer (PC), a tablet PC, a set-top box (STB), a Personal

Digital Assistant (PDA), a mobile telephone, a web appliance, a network router, switch or bridge, or any machine capable of executing instructions (sequential or otherwise) specifying actions to be taken (e.g., performed) by the machine 400. Further, while only a single machine 400 is illustrated, the term “machine” shall also be taken to include any collection of machines that individually or jointly execute a set (or multiple sets) of instructions to perform any one or more of the methodologies discussed herein.

Example machine (e.g., computer system) 400 can include a processor 402 (e.g., a central processing unit (CPU), a graphics processing unit (GPU) or both), a main memory 404 and a static memory 406, some or all of which can communicate with each other via a bus 408. The machine 400 can furtt1er include a display unit 410, an alphanumeric input device 412 (e.g., a keyboard), and a user interface (UI) navigation device 411 (e.g., a mouse). In an example, the display unit 810, input device 417 and UI navigation device 414 can be a touch screen display. The mact1ine 400 can additionally include a storage device (e.g., drive unit) 416, a signal generation device 418 (e.g., a speaker), a network interface device 420, and one or more sensors 421, such as a global positioning system (GPS) sensor, compass, accelerometer, or other sensor.

The storage device 416 can include a machine readable medium 422 on which is stored one or more sets of data structures or instructions 424 (e.g., software) embodying or utilized by any one or more of the methodologies or functions described herein. The instructions 424 can also reside, completely or at least partially, within the main memory 404, within static memory 406, or within the processor 402 during execution thereof by the machine 400. In an example, one or any combination of the processor 402, the main memory 404, the static memory 406, or the storage device 416 can constitute machine readable media.

While the machine readable medium 422 is illustrated as a single medium, the term “machine readable medium” can include a single medium or multiple media (e.g., a centralized or distributed database, and/or associated caches and servers) that configured to store the one or more instructions 424. The term “machine readable medium” can also be taken to include any tangible medium that is capable of storing, encoding, or carrying instructions for execution by the machine and that cause the machine to perform any one or more of the methodologies of the present disclosure or tt1at is capable of storing, encoding or carrying data structures utilized by or associated with such instructions. The term “machine readable medium” can accordingly be taken to include, but not be limited to, solid-state memories, and optical and magnetic media. Specific examples of mact1ine readable media can include non-volatile memory, including, by way of example, semiconductor memory devices (e.g., Electrically Programmable Read-Only Memory (EPROM), Electrically Erasable Programmable Read-Only Memory (EEPROM)) and flash memory devices; magnetic disks such as internal hard disks and removable disks; magneto-optical disks; and CD-ROM and DVD-ROM disks.

The instructions 424 can further be transmitted or received over a communications network 426 using a transmission medium via the network interface device 420 utilizing any one of a number of transfer protocols (e.g., frame relay, IP, TCP, UDP, HTTP, etc.). Example communication networks can include a local area network (LAN), a wide area network (WAN), a packet data network (e.g., the Internet), mobile telephone networks (e.g., cellular networks), Plain Old Telephone (POTS) networks, and wireless data networks (e.g., IEEE 802.11 standards family known as Wi-Fi®, IEEE 802.16 standards family known as WiMax®), peer-to-peer (P2P) networks, among ott1ers. The term “transmission medium” shall be taken to include any intangible medium that is capable of storing, encoding or carrying instructions for execution by the machine, and includes digital or analog communications signals or other intangible medium to facilitate communication of such software.

### II. KITS AND PACKAGING

Described herein are kits comprising one or more primer or probes for detecting one or more polymorphisms in genes described above, in addition to containers for storage and/or use, and instructions for use. The kit can be a package which houses a container which contains compounds of the disclosure or formulations of the disclosure and also houses instructions for performing methods as described herein on a subject. The disclosure further relates to a commercial package comprising one or more primers or probes of the disclosure or formulations of the disclosure together with instructions for simultaneous, separate or sequential use.

The disclosure also provides a sample collection device comprising one or more containers or devices that are capable of collecting samples as described herein for diagnostic analysis, for example, urine or spot urine samples. Associated with such container(s) can be various written materials such as instructions for use, or a notice in the form prescribed by a governmental agency regulating the labeling, manufacture, use or sale of aspects of the kit, which notice reflects approval by the agency of manufacture, use, or sale for human administration.

The disclosure also relates to articles of manufacture and kits containing materials useful for determining a salt index disclosed herein. An article of manufacture may comprise a container with a label. Examples of suitable containers include bottles, vials, and test tubes, or a collection device such as a urine or spot urine collector, which may be formed from a variety of materials including paper, glass, plastic, and other polymers. A container holds compositions of the disclosure which are effective for determining a salt index of a subject or can hold or otherwise collect a sample from the subject. The label on the container indicates that the compounds of the disclosure or formulations of the disclosure are used for determining an individual's salt index disclosed herein and may also indicate directions for use. In aspects of the disclosure, a medicament or formulation in a container may comprise any of the medicaments or formulations disclosed herein. Examples of urine collection devices are well known in the art, but some include, for example, the BD Vacutainer Urine Collection Cup and others. Urine may be in liquid form, or may otherwise be dried or on a paper-or polymer-based spot collection device.

The disclosure also contemplates kits comprising one or more primers or probes of the disclosure. In aspects of the disclosure, a kit of the disclosure comprises a container described herein. In particular aspects, a kit of the disclosure comprises a container described herein and a second container comprising a buffer. A kit may additionally include other materials desirable from a commercial and user standpoint, including, without limitation, buffers, diluents, filters, needles, syringes, and package inserts with instructions for performing any methods disclosed herein (e.g., methods for treating a disease disclosed herein). A medicament or formulation in a kit of the disclosure may comprise any of the formulations or compositions disclosed herein.

The compositions (i.e., those comprising, consisting essentially of, or consisting of probes or primers described herein) can be utilized in the preparation of a kit. In some embodiments, kits are provided for carrying out any of the methods described herein. The kits of this disclosure may comprise a carrier container being compartmentalized to receive in close confinement one or more containers such as vials, tubes, and the like, each of the containers comprising one of the separate elements to be used in the methods.

In some instances, one of the containers may comprise a composition as described in this disclosure that is, or can be, detectably labeled. The kit may also have containers containing buffer(s) and/or a container comprising a reporter-means, such as a biotin-binding protein, such as avidin or streptavidin, bound to a reporter molecule, such as an enzymatic or fluorescent label. In some embodiments, the kit comprises separate containers containing compositions described herein and a detectable label.

A protocol as described in this disclosure for use in determining a salt index in subjects may be delivered in a pharmaceutical package or kit to doctors and patients with blood pressure issues in particular. Such packaging is intended to improve patient convenience and compliance with the treatment plan. Typically, the packaging comprises paper (cardboard) or plastic. In some embodiments, the kit or pharmaceutical package further comprises instructions for use (e.g., for administering according to a method as described herein).

In some embodiments, a pharmaceutical package or kit comprises unit dose forms of a composition or components of compositions described herein. In some embodiments, the pharmaceutical package or kit further comprises unit dose forms of one or more of an additional therapeutic, for example, another medicament used for treatment of a disorder in a patient.

### III. METHODS OF TREATMENT

As described herein, the present disclosure provides a method of treating a subject with hypertension, salt-sensitivity, salt-resistance, inverse salt-sensitivity, or any combination of any thereof. Aims of methods of treatment described herein are to help normalize (i.e., return or keep a subject in or around a normotensive state) a subject's blood pressure, in particular, in response to a salt challenge. Methods of treatment as described herein utilize a salt-sensitivity determination of an individual to help a physician or other healthcare provided made informed choices about medicaments that can be administered or otherwise prescribed to an individual in order to minimize any potential cardiovascular complications that could arise from either the individuals phenotype, treatment regime, or both. Methods as described herein can involve prescription of a low salt or high salt diet, small molecules (i.e., less than 2500 daltons), or other therapeutics contemplated by the present disclosure.

The compositions described herein are useful in, inter alia, methods for managing or otherwise normalizing blood pressure in a subject. In some embodiments, the subject has or is suspected to have a hypertension, or blood pressure that is otherwise affected by salt intake. In some embodiments, the subject is diagnosed with essential hypertension. In some embodiments, the subject is a human that is suspected of having a aberrant blood pressure that is salt-sensitive.

The compositions can be administered to a subject, e.g., a human subject, using a variety of methods that depend, in part, on the route of administration. The route can be, e.g., intravenous injection or infusion (IV), subcutaneous injection (SC), intraperitoneal (IP) injection, intramuscular injection (IM), intradermal injection (ID), subcutaneous, transdermal, intracavity, oral, or intrathecal injection (IT). The injection can be in a bolus or a continuous infusion. Techniques for preparing injectate or infusate delivery systems containing therapeutics are well known to those of skill in the art. Generally, such systems should utilize components which will not significantly impair the biological properties of the therapeutics, such as the ability to interact with a receptor or transporter (see, for example, Remington's Pharmaceutical Sciences, 18th edition, 1990, Mack Publishing). Those of skill in the art can readily determine the various parameters and conditions for producing therapeutic injectates or infusates without resort to undue experimentation.

Treating or treatment of any disease or disorder refers to ameliorating a disease or disorder that exists in a subject or a symptom thereof, in particular, ameliorating symptoms of aberrant (i.e., non-normotensive) blood pressure. The term ameliorating refers to any therapeutically beneficial result in the treatment of a disease state, e.g., a normalization of blood pressure or improved survival.

Thus, treating or treatment includes ameliorating at least one parameter or symptom. Treating or treatment includes modulating the disease or disorder, either physically (e.g., stabilization of a discernible symptom) or physiologically (e.g., stabilization of a physical parameter) or both. Thus, in the disclosed methods, treatment can refer to a 10%, 20%, 30%, 40%, 50%, 60%, 70%, 80%, 90%, or 100% reduction in the severity of an established disease or condition or symptom of the disease or condition. For example, a method for treating aberrant blood pressure that may be salt sensitive, resistant, or inverse salt-sensitive in a subject by administering a composition as described in this disclosure is considered to be a treatment or therapeutic, for example, if there is a 10% improvement according to the measured or observed parameter in a subject as compared to a control. Thus, the reduction can be a 10%, 20%, 30%, 40%, 50%, 60%, 70%, 80%, 90%, 100%, or more (or any percent improvement in between 10% and 100%) as compared to native or control levels. It is understood that treatment does not necessarily refer to a cure or complete ablation of the disease, condition, or symptoms of the disease or condition.

Described herein are therapeutically effective amounts as defined previously. A therapeutically effective amount is not, however, a dosage so large as to cause adverse side effects. A suitable dose capable of managing blood pressure or normalizing blood pressure in a subject, can depend on a variety of factors including the particular construct used and whether it is used concomitantly with other therapeutic agents. Generally, a therapeutically effective amount may vary with the subject's age, condition, and sex, as well as the extent of the disease in the subject and can be determined by one of skill in the art. Other factors can include, e.g., other medical disorders concurrently or previously affecting the subject, the general health of the subject, the genetic disposition of the subject, diet, time of administration, rate of excretion, drug combination, and any other additional therapeutics that are administered to the subject. It should also be understood that a specific dosage and treatment regimen for any particular subject also depends upon the judgment of the treating medical practitioner (e.g., doctor or nurse). A therapeutically effective amount is also one in which any toxic or detrimental effects of the composition are outweighed by the therapeutically beneficial effects. The dosage of the therapeutically effective amount may be adjusted by the individual physician or veterinarian in the event of any complication.

A pharmaceutical composition can include a therapeutically effective amount of any one or more therapeutics described herein. Such effective amounts can be readily determined by one of ordinary skill in the art as described above, embodiments are given throughout the present disclosure.

If unknown, suitable human doses of any of the therapeutics described herein can further be evaluated in, e.g., Phase I dose escalation studies. See, e.g., van Gurp et al. (2008) Am J Transplantation 8 (8): 1711-1718; Hanouska et al. (2007) Clin Cancer Res 13 (2, part 1): 523-531; and Hetherington et al. (2006) Antimicrobial Agents and Chemotherapy 50 (10): 3499-3500.

Toxicity and therapeutic efficacy of such therapeutics can be determined by known pharmaceutical procedures in cell cultures or experimental animals (e.g., animal models of any of the cancers described herein). These procedures can be used, e.g., for determining the LD₅₀ (the dose lethal to 50% of the population) and the ED₅₀ (the dose therapeutically effective in 50% of the population). The dose ratio between toxic and therapeutic effects is the therapeutic index, and it can be expressed as the ratio LD₅₀/ED₅₀. A compositions comprising therapeutics that exhibits a high therapeutic index is preferred. While constructs that exhibit toxic side effects may be used, care should be taken to design a delivery system that targets such constructs to the site of affected tissue and to minimize potential damage to normal cells and, thereby, reduce side effects.

The data obtained from the cell culture assays and animal studies can be used in formulating a range of dosage for use in humans. The dosage of a therapeutic lies generally within a range of circulating concentrations of the therapeutics that include the ED₅₀ with little or no toxicity. The dosage may vary within this range depending upon the dosage form employed and the route of administration utilized. For therapeutics herein, the therapeutically effective dose can be estimated initially from cell culture assays (in particular, assays utilizing RPTCs, in particular, RPTCs derived from the subject. A dose can be formulated in animal models to achieve a circulating plasma concentration range that includes the EC₅₀ (i.e., the concentration of the construct—e.g., antibody—which achieves a half-maximal inhibition of symptoms) as determined in cell culture. Such information can be used to more accurately determine useful doses in humans. Levels in plasma may be measured, for example, by high performance liquid chromatography. In some embodiments, e.g., where local administration is desired, cell culture or animal models can be used to determine a dose required to achieve a therapeutically effective concentration within the local site.

In some embodiments, a composition or component of a composition described herein comprising a therapeutic described herein can be administered to a subject as a monotherapy. Alternatively, the composition or component of a composition described herein or therapeutic can be administered in conjunction with other therapies for blood pressure management (combination therapy). For example, the composition can be administered to a subject at the same time, prior to, or after, a second therapy. In some embodiments, the composition or component of a composition described herein, and the one or more additional active agents are administered at the same time. Optionally, the composition or component of a composition described herein is administered first in time and the one or more additional active agents are administered second in time. In some embodiments, the one or more additional active agents are administered first in time and the therapeutic composition or component of a composition described herein is administered second in time. Optionally, the composition or component of a composition described herein, and the one or more additional agents are administered simultaneously in the same or different routes.

A composition as described herein can replace or augment a previously or currently administered therapy, such as previously prescribed blood pressure management medicament.

Monitoring a subject (e.g., a human patient) for an improvement of symptoms as described herein, for example, normalization of blood pressure following a salt challenge, means evaluating the subject for a change in a given parameter or symptom exhibited by the subject by a clinician in a social setting or self-reporting by the patient. In some embodiments, the evaluation is performed at least one (1) hour, e.g., at least 2, 4, 6, 8, 12, 24, or 48 hours, or at least 1 day, 2 days, 4 days, 10 days, 13 days, 20 days or more, or at least 1 week, 2 weeks, 4 weeks, 10 weeks, 13 weeks, 20 weeks or more, after an administration. The subject can be evaluated in one or more of the following periods: prior to beginning of treatment; during the treatment; or after one or more elements of the treatment have been administered. Evaluation can include evaluating the need for further treatment, e.g., evaluating whether a dosage, frequency of administration, or duration of treatment should be altered. It can also include evaluating the need to add or drop a selected therapeutic modality.

In certain embodiments, the effective amount of a pharmaceutical composition comprising one or more therapeutics of the present disclosure to be employed therapeutically depends, for example, upon the therapeutic context and objectives. One skilled in the art will appreciate that the appropriate dosage levels for treatment, according to certain embodiments, vary depending, in part, upon the molecule delivered, the indication for which a treatment is being used, the route of administration, and the size (body weight, body surface or organ size) and/or condition (the age and general health) of the patient. The clinician can titer the dosage and modify the route of administration to obtain the optimal therapeutic effect.

The clinician also selects the frequency of dosing, taking into account the pharmacokinetic parameters of the active components in the formulation used. Such pharmacokinetic parameters are well known in the art, i.e., the rate of absorption, bioavailability, metabolism, clearance, and the like (see, e.g., Hidalgo-Aragones (1996) J. Steroid Biochem. Mol. Biol. 58:611-617; Groning (1996) Pharmazie 51:337-341; Fotherby (1996) Contraception 54:59-69; Johnson (1995) J. Pharm. Sci. 84:1144-1146; Rohatagi (1995) Pharmazie 50:610-613; Brophy (1983) Eur. J. Clin. Pharmacol. 24:103-108; the latest Remington's, supra). In certain embodiments, a clinician administers the composition until a dosage is reached that achieves the desired effect. In certain embodiments, the composition can therefore be administered as a single dose or as two or more doses (which may or may not contain the same amount of the desired molecule) over time, or as a continuous infusion via, for example, an implantation device or catheter. Further refinement of the appropriate dosage is routinely made by those of ordinary skill in the art and is within the ambit of tasks routinely performed by them. In certain embodiments, appropriate dosages can be ascertained through use of appropriate dose-response data.

In certain examples, the compositions thereof can be administered once every other day at least four times. An exemplary treatment regime may include administration once per day, once per week, twice a week, once every two weeks, once every three weeks, once every four weeks, once a month, once every 3 months, or once every three to 6 months. In some cases, the treatment comprises administering a composition according to one of the aforementioned dosing regimens for a first period and another of the aforementioned dosing regimens for a second period. In some cases, the treatment discontinues for a period of time before the same or a different dosing regimen resumes. For example, a patient may be on a dosing regimen for two weeks, off for a week, on for another two weeks, and so on.

i. Exemplary Pharmacological Treatments

The detection of the SNPs, as well as blood pressure measurements following salt diets, described herein for use in determining an individuals personal salt index can be further used to help establish a treatment regimen and for monitoring treatment. One of ordinary skill in the art will appreciate that a variety of drugs, or combinations of drugs, can be used based in the age, health, and other characteristics of the subject. A diet comprised of salt concentrations or amounts that do not exceed each subject's personal salt index can also be established to control sodium intake.

For example, a subject found to be sensitive can be treated with one or more diuretics, mineralocorticoid receptor antagonists, beta-blockers, alpha-blockers, ACE inhibitors, angiotensin II receptor blockers, calcium channel blockers, central agonists, peripheral-acting adrenergic blockers, direct vasodilators, or direct renin inhibitors. Use of these drugs can also be coupled with a diet to limit sodium intake.

The present disclosure further encompasses, in embodiments, the use of drugs such as fenofibrate, which has been recently shown to lower blood pressure in salt-sensitive but not salt resistant hypertension (Gilbert et al., 2013, J. Hypertension).

The types of drugs, and some specific drugs, useful according to methods of the present disclosure include, but are not limited to:

Diuretics, including but not limited to, for example: Aldactone (spironolactone), Dyrenium (triamterene), Esidrix, Hydrodiuril, and Microzide (hydrochlorothiazide or HCTZ), Hygroton and Thalitone (chlorthalidone), Lasix (furosemide), Lozol (indapamide), Midamor (amiloride hydrochloride), and Mykrox and Zaroxolyn (metolazone).

Combination diuretics are also encompassed by the methods of the invention and include, but are not limited to: Aldactazide (spironolactone and hydrochlorothiazide), Dyazide and Maxzide (hydrochlorothiazide and triamterene), and Moduretic (amiloride hydrochloride and hydrochlorothiazide).

Beta-Blockers, including but not limited to, for example: Blocadren (timolol), Cartrol (carteolol hydrochloride), Coreg (carvedilol), Corgard (nadolol), Inderal (propranolol), Kerlone (betaxolol), Levatol (penbutolol sulfate), Lopressor and Toprol XL (metoprolol), Sectral (acebutolol), Tenormin (atenolol), Visken (pindolol), Zebeta (bisoprolol fumarate), Normodyne and Trandate (labetolol),

Alpha-Blockers, including but not limited to, for example: Cardura (doxazosin), Hytrin (terazosin), and Minipress (prazosin).

ACE Inhibitors: Angiotensin-converting enzyme inhibitors (ACE) are high blood pressure medications that prevent production of angiotensin II. Examples of ACE inhibitors include, but are not limited to: Accupril (quinapril), Altace (ramipril), Capoten (captopril), Mavik (trandolapril), Lotensin (benazepril), Monopril (fosinopril), Prinivil and Zestril (lisinopril), Univasc (moexipril), and Vasotec (enalapril).

ARBs (Angiotensin II Receptor Blockers, i.e., AT₁R). Examples of ARBs, include, but are not limited to, for example: Atacand (candesartan), Avapro (irbesartan), Benicar (olmesartan), Cozaar (losartan), Diovan (valsartan), Micardis (telmisartan), Teveten (eprosartan), losartan, and telmisartan.

Calcium Channel Blockers (CCBs). Examples of CCBs include, but are not limited to, for example: Adalat and Procardia (nifedipine), Calan, Covera, Isoptin, Verelan, and others (verapamil), Cardene (nicardipine), Cardizem, Cartia, Dilacor, and Tiazac (diltiazem), DynaCirc (isradipine), Norvasc (amlodipine), Plendil (felodipine), and Sular (nisoldipine).

Central Agonists. These medications target receptors. Examples of central agonists include, but are not limited to, for example: Aldomet (methyldopa), Catapres (clonidine), Tenex (guanfacine), and Wytensin (guanabenz).

Peripheral-Acting Adrenergic Blockers. Examples of peripheral-acting adrenergic blockers include, but are not limited to, for example: Hylorel (guanadrel), Ismelin (guanethidine), and Serpasil (reserpine).

Direct Vasodilators. Examples of direct vasodilators include, but are not limited to, for example: Apresoline (hydralazine), and Loniten (minoxidil).

Direct Renin Inhibitors. Direct renin inhibitors, ACE inhibitors, and ARBs all target the same process that narrows blood vessels. However, each type of medication blocks a different part of the process. Direct renin inhibitors block the enzyme renin from triggering a process that helps regulate blood pressure. Tekturna (aliskiren) is a direct renin inhibitor. Tekturna can be used alone or in combination with a diuretic or other medicines for high blood pressure.

The dosage of the active compound(s) being administered will depend on the condition being treated, the particular compound, and other clinical factors such as age, sex, weight, and health of the subject being treated, the route of administration of the compound(s), and the type of composition being administered (tablet, gel cap, capsule, solution, suspension, inhaler, aerosol, elixir, lozenge, injection, patch, ointment, cream, etc.). It is to be understood that the present invention has application for both human and veterinary use.

For example, in one embodiment relating to oral administration to humans, a dosage of between approximately 0.1 and 300 mg/kg/day, or between approximately 0.5 and 50 mg/kg/day, or between approximately 1 and 10 mg/kg/day, is generally sufficient, but will vary depending on such things as the disorder being treated, the length of treatment, the age, sex, weight, and/or health of the subject, etc. The drugs can be administered in formulations that contain all drugs being used, or the drugs can be administered separately. In some cases, it is anticipated that multiple doses/times of administration will be required or useful. The present invention further provides for varying the length of time of treatment.

### IV. EXAMPLES

Now having described the embodiments of the disclosure, in general, the examples describe some additional embodiments. While embodiments of the present disclosure are described in connection with the example and the corresponding text and figures, there is no intent to limit embodiments of the disclosure to these descriptions. On the contrary, the intent is to cover all alternatives, modifications, and equivalents included within the spirit and scope of embodiments of the present disclosure.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is in atmosphere. Standard temperature and pressure are defined as 25° C. and 1 atmosphere.

### Example 1

### A. Introduction

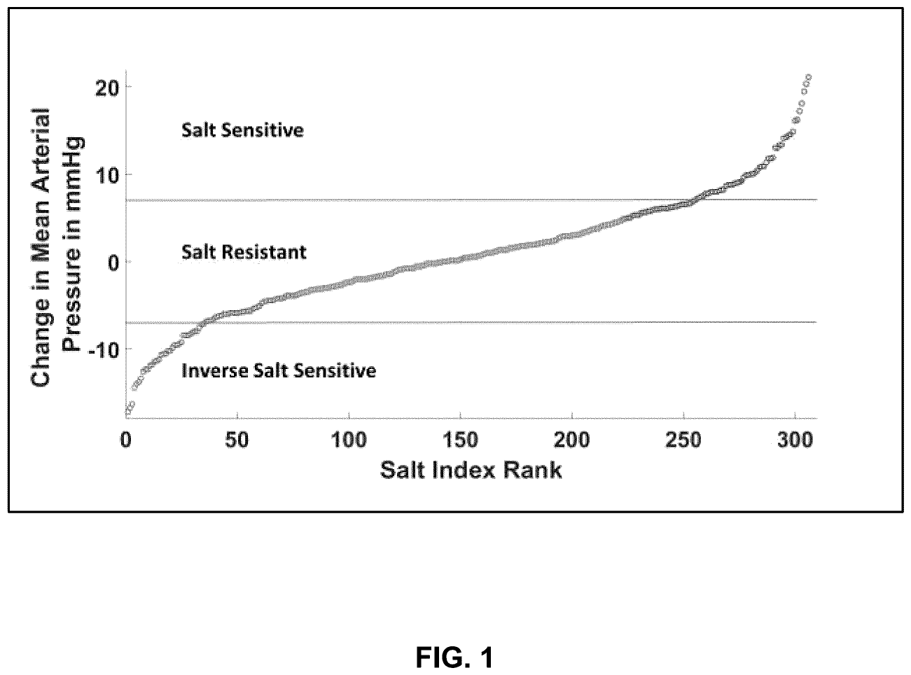

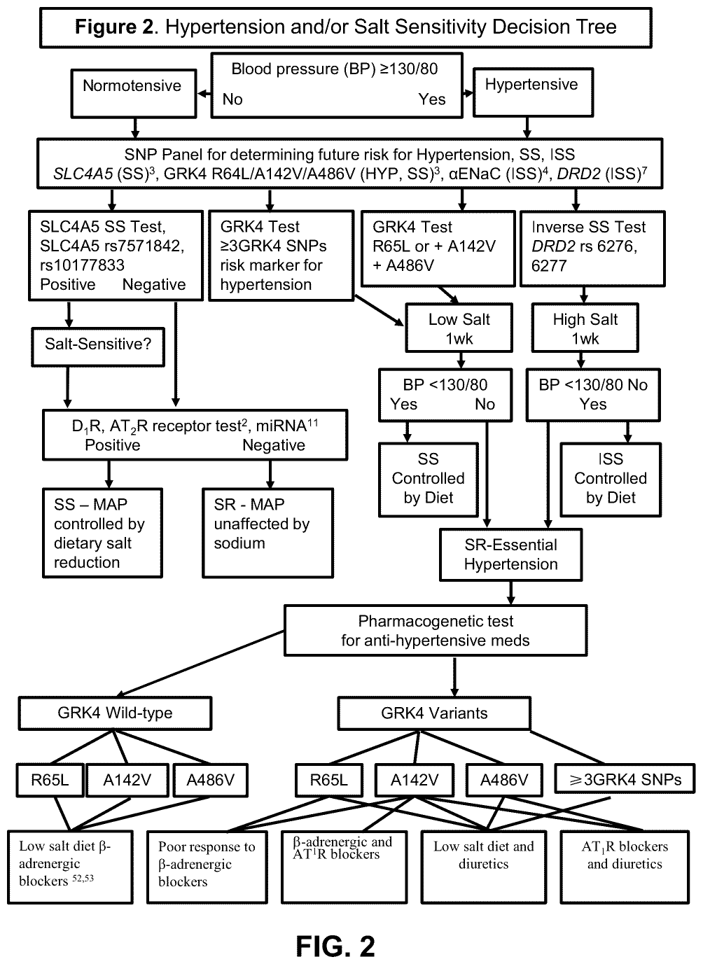

Cardiovascular disease can result from continuously consuming sodium, as sodium chloride, at an amount that cannot be excreted properly by an individual's personal capability.¹ This example summarizes what is known about the individual variability of the blood pressure (BP) responses to changes in sodium intake. Controlled diet studies found that the BP responses to sodium intake in humans are trinary. Approximately 60% (N=184/306) had less than 7 mm Hg change in mean arterial pressure (MAP) when sodium intake was changed from high sodium intake 300 mmol/day (6,900 mg, equivalent to 17,550 mg NaCl) to low sodium intake 10 mmol (230 mg/day, equivalent to 585 mg NaCl) and were termed salt-resistant individuals (SR).² Another group of approximately 20% (N=54/306) had an increase in MAP when the sodium intake was changed to high sodium intake or vice versa (termed salt-sensitive, SS).^{2,3} However, when the effect of sodium intake on MAP was examined as the change in MAP for each individual (FIG. 1), approximately 11% (N=34/306) of individuals had a paradoxical change in MAP, i.e., the change in MAP was opposite to the directional change in MAP in SS individuals.² Their MAPs increased when sodium intake was reduced from the high sodium diet to the low sodium diet and had the opposite effect of increasing MAP when sodium intake was reduced. Since high sodium intake reduced their MAP, these individuals were classified as inverse salt-sensitive (ISS).² Others have reported that a low sodium (2,645 mg/day) diet can increase BP in some individuals even to hypertensive levels leading to increased mortality and morbidity. 4-6 Making comparisons among studies is difficult since no consensus on salt dietary protocols or agreement about the MAP cutoff to use to define each group. However, the distribution of the trinary response to sodium intake, i.e. SR, SS, and ISS has been justified.² A salt intervention study in a healthy population showed that a low sodium intake (920 mg/day) increased daytime systolic blood pressure in 20.8% of 465 study participants.7 In a recent study, it was found that there is a greater prevalence of ISS among non-obese individuals, and this characteristic is not influenced by age.⁸ Notably, plasma osmolality levels increased significantly in both SR (p<0.001) and SS (p<0.001) individuals when transitioning from low to high sodium intake, but did not change significantly in ISS.⁸ These findings suggest that ISS individuals tend to excrete sodium more efficiently compared to their SR and SS counterparts.⁹ This leads to a crucial question: Do ISS individuals exhibit a higher risk of cardiovascular disease compared to those SR individuals? A recent retrospective re-evaluation of a 23-year salt study involving 844 naïve hypertensive patients followed for about 16 years showed that the incidence of cardiovascular events 5.94 time higher in ISS than SR individuals.¹⁰ Severe hypertensive target organ damage was 10-fold higher in SS and over 15-fold higher in ISS than SR individuals.¹⁰ Determination of the cause of the wide variability in BP with sodium intake continues to be investigated, but the variable response to sodium is complicated by the fact that BP is influenced by environment, genetics, epigenetics and behavior.¹¹ Based on studies of the Inventor and those of others, a mosaic model¹² may demonstrate the many genetic loci where sodium regulation is altered leading to an individual's MAP set point and ultimately the probability of cardiovascular disease.¹³⁻¹⁵ Also discussed herein is the diagnosis of ISS and potential therapeutic interventions to treat SS and ISS individuals which are summarized in a decision tree format (FIG. 2) to encourage physicians to provide personalized sodium intake recommendations to their patients.

### B. Mechanisms and Pathophysiology

Many organs participate in regulating sodium balance including the kidney, gastrointestinal tract, respiratory tract, and skin.^{1,2} There are numerous sodium transporters, exchangers, channels, and pumps in the intestines and kidney that regulate the ability of the gastrointestinal cells to absorb ions from the ingested food and renal tubules to reabsorb ions from the glomerular filtrate and back into circulation.^{16,17} The amount of intestinal absorption and renal reabsorption of sodium is dictated not only by the amount of sodium that is consumed but also by each individual's coding DNA and epigenetic modulations that control the action of sodium transporters, exchangers, channels, and pumps. If single nucleotide polymorphisms (SNPs) are present in these genes that regulate their expression and/or function, they can downregulate or upregulate the transmembrane flux of ions imparting a genetic predisposition for the increased BP in SS and ISS individuals.¹⁸ For example, in clinical studies focused on salt sensitivity, it was discovered polymorphisms in the sodium-bicarbonate cotransporter NBCe2 located in the luminal membrane of the renal proximal tubule coded by SLC4A5 (rs7571842 and rs10177833) that were highly associated with the SS phenotype but not the SR or ISS phenotype. These SNPs remained significant after adjusting for BMI and age, (P=8.9×10⁻⁵ and 2.6×10⁴ and odds ratios 0.210 and 0.286, respectively).³ These studies were replicated in a sodium diet protocol conducted by Williams et al (rs7571842 (P=1.2×10-5); rs1017783 (P=1.1×10-4).³

ISS individuals also have polymorphisms of genes that are associated with their phenotype. Reduced αENaC expression but increased activity in ISS is caused by the ENaC variant rs4764586, which is more prevalent in inverse salt sensitivity.¹⁹ It was also found that trypsin-activated ENaC activity was higher in RPTCs from ISS that SR individuals. This could at least partially explain the paradoxical increase in BP with low sodium diet in ISS. By contrast, under high salt conditions ISS individuals could be protected from increased BP by the lower renal ENaC expression which may lead to greater natriuresis, relative to SS and SR individuals.¹⁹

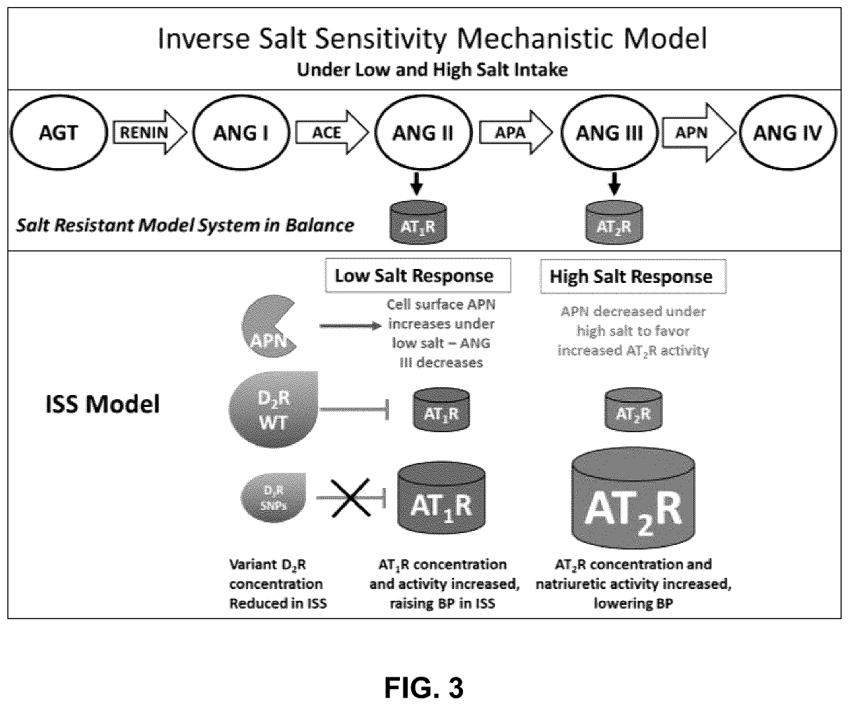

An important regulator of BP is the renin-angiotensin system (RAS) as represented in the model of various renin-angiotensin system (RAS) components that contribute to sodium balance (FIG. 3). The angiotensin (Ang) type 1 receptor (AT₁R) is an antinatriuretic G protein-coupled receptor that increases calcium signaling via Ang II in ISS.20 Enzymes that convert RAS peptides into downstream mediators of sodium balance may also play a role in the pathogenesis of ISS. In ISS, increased Aminopeptidase N (APN) converts Ang III into Ang IV reducing the concentration of Ang III that stimulates the Ang II type 2 receptor (AT₂R).²¹ The AT₂R is an important mediator of the decrease in renal tubular sodium reabsorption, which results in an increase in urinary sodium excretion.²² The AT₂R also interacts with D₃R to decrease renal sodium transport and increase sodium excretion.²³ Overlack found that a low sodium diet (460 mg sodium/day) caused a greater increase in plasma renin activity in “counter regulators” (aka inverse salt sensitives) than SR and SS humans.²⁴ These observations indicate that ISS study participants on a low salt intake have an overstimulated RAS that could contribute to their high BP on very low sodium intake. The dopamine type 2receptor (D2R), dopamine type 3 receptor (D3R), and dopamine type 4 receptor (D4R) are involved in inhibiting AT₁R in the kidney.²⁵⁻²⁷ The DRD2 gene variants, rs6276 and rs6277, were found to be associated with an increase in BP on a low sodium (10 mmol/day) diet.²⁸ Plasma membrane D2R expression was measured using an extracellular epitope-specific rabbit anti-D2R antibody and found to be reduced by 25.3+0.5% in RPTCs from ISS subjects (p<0.01, N=5/group), relative to RPTCs from SR subjects.²

There are additional genes associated with ISS but less studied than those above. These genes and their translated proteins could also serve as additional diagnostic markers in the diagnosis of SS and ISS. For example, SMIT1 (SLC5A3) and SMIT2 (SLC5A11, SGLT6) are two sodium/myoinositol cotransporters that are expressed in the human kidney, but SMIT2 is responsible for the RPT apical sodium/myoinositol transport.29 SLC5A11rs11074656 is associated with ISS.30 In subjects carrying both SLC5A11 and DRD2 SNPs, those with more total SNPs have higher increase in MAP on low salt diet than on high salt diet.²⁸ As with the DRD2 rs6276 and rs6277 SLC5A11 protein expression is decreased by SLC5A11 rs11074656.28

DNA regulators such as micro-RNAs (miRNAs) are beginning to serve as disease markers.³¹ The association of urinary micro-RNAs with an individual's BP response to dietary sodium intake has been reported.³² A microarray containing 1898 probes was used to study the human urinary exosomal miRNome; of 194 miRNAs 45 miRNAs had significant associations with SS or ISS.³³ A total of 25 miRNAs were found to be associated with ISS.³³

Although not differentially expressed, miR-485-5p is one of the 194 miRNAs found, which binds to the DRD2 rs6276, reducing D₂R expression.34 In human RPT cells, miR-217 which mediates the protective effects of D₂R on fibrosis of human RPTCs is regulated by DRD2; DRD2 rs6276 reduces D₂R expression.³³ Furthermore, a miRNA mimic that behaved like the endogenous mir-485-5p miRNA reduced the expression of D₂R, but only in the RPTCs from the urine of individuals with ISS.³³ A miRNA blocker inhibited endogenous miRNAs, resulting in normalization of the expression of the D₂R.²⁸

The G protein-coupled receptors dopamine type 1 receptor (DIR) and the AT₂R increase the excretion of sodium (natriuretic) while αENaC and RAS aid in the renal tubular reabsorption of sodium (antinatriuretic).³⁵⁻³⁷ The relative abundance and/or activity of these receptors and channels in RPTCs have been found to correlate with the change in BP following a change in sodium intake that can serve as diagnostic tests for ISS, SR and SS.^{13,19,38} Polymorphisms in these receptors and channels are also associated with the expression of the ISS and SS phenotypes.¹³ A three-pronged approach to determining the sodium excretion set point or Personal Salt Index of an individual according to the present example include: 1) genes associated with both salt sensitivity and inverse salt sensitivity; 2) cellular DIR and AT₂R recruitment following a sodium challenge in living kidney cells; and 3) relative abundance of exosomal miRNAs that are excreted in the urine.

### C. Clinical Implications

Although the dietary sodium recommendation by the Institute for Medicine is intended as a ‘one size fits all’ recommendation, it is becoming clear that each individual is genetically programmed with a “Personal Salt Index” and so dietary salt (NaCl) consumption guidelines should be personalized.^{4,13, 39-42} Salt intake and cardiovascular morbidity and mortality have a J-shaped curve relationship, in that a low-salt diet as well as a high salt diet is associated with increased risk for stroke, kidney failure, blindness, and heart attack. The relative risk of developing cardiovascular events is 12.67 times higher in SS than SR and 5.94 times higher in ISS than SR individuals.¹⁰ Severe hypertensive target organ damage is 10-fold higher in SS compared to SR and 15-fold higher in ISS compared to SR.10 In one study individuals with the lowest ⅓rd of measured 24 hour urine sodium measured were the individuals with the highest cardiovascular deaths.⁴⁰ Thus there is a compelling need to classify individual patients as ISS, SR, and SS. However, the current research diet protocols are inconvenient and expensive for routine patient care. Novel diagnostic tests could be conveniently measured for each patient during their annual routine physical exam. If these guidelines are followed, the incidence of early death from cardiovascular crises could be significantly reduced by approximately 60%.^{43,44} To assist with implementing a personalized diagnosis we suggest a decision tree approach (FIG. 2) could be considered that involves genetic analysis with published association data^{33,45} and RPT measurements performed on a spot urine.^{13,38,46} Ultimately, patients could follow a controlled sodium diet in order to avoid antihypertensive medication if they are not challenged with dietary compliance or don't have underlying essential hypertension.

The decision tree depicted in FIG. 2 begins with a blood pressure measurement carefully following established guidelines. For example, BP cuff size is important to prevent aberrant readings.⁴⁷ This decision tree identifies individuals who are hypertensive due to an aberrant balance between their natriuretic and anti-natriuretic renal sodium metabolism leading to sodium retention and SS13, and those who have hypertension due to insufficient sodium in their diet (ISS).³⁸ We and others have determined that approximately 31% of patients will present with either SS or ISS.2 The right side of the decision tree in FIG. 2 identifies individuals who are hypertensive due to aberrant balance between their natriuretic and antinatriuretic renal sodium metabolism, and those who have hypertension due to insufficient sodium in their diet (ISS=˜15% of the population).^{2,48,49} The extreme right pathway will cull out individuals with the ISS phenotype since the DRD2 SNP at rs6276 is associated with ISS.³ If this DRD2 SNP is present and the individual's blood pressure normalizes on a high salt diet then the essential hypertension phenotype can be ruled out. The treatment for this individual would be a gradual increase in dietary sodium until their blood pressure normalizes. The decision tree also contains a step to perform the diagnostic tests using living RPTC obtained from urine specimens phenotyped for ISS, SS, and SR.^{13,38} If the blood pressure does not normalize on the high sodium intake, then one can assume the individual has hypertension insensitive to salt and that their hypertension might be better controlled by medication. The pharmacogenetic data should then be applied since it might shorten the effort required to prescribe appropriate therapeutics (FIG. 2, lower right side). If an individual carries SNPs in GRK4 (R65L, A142V, A486V then they are at risk for either hypertension or hypertension that is salt sensitive.

The GRK4 polymorphisms R65L, A142V, and A486V have been linked to various pharmacologic and sodium intake interventions in published studies considering drug intake, race, age, and renal function.^{35,50-52} For example, if a subject is hypertensive and does not express any polymorphisms in GRK4 that are associated with hypertension (R65L, A142V, A486V) then they have a higher likelihood of reducing their BP on a low sodium intake 53 and/or β-adrenergic α-adrenergic receptor blockers.^{54,55} If an individual expresses only the R65L polymorphism then they are likely to express lower BP on a low sodium intake or sodium-eliminating diuretics, but not a-adrenergic receptor blockers.^{54,55} Expression of only A142V results in the favorable response to all treatment options, and expression of A486V does not cause a favorable response to all the routine treatment options shown. Individuals expressing 3 or more GRK4 polymorphisms at any of the 6 total GRK4 alleles, then they are more likely to respond to a low salt diet and/or a diuretic.⁵⁶

The left side of the decision tree is for individuals who present with normal blood pressure as defined as a BP less than 130/80 mm Hg systolic/diastolic. These normotensive individuals may still be salt sensitive in that their pressure increases greater than 7 mm Hg on a high salt diet. Individuals who have SNPs in the sodium bicarbonate co-transporter (expressed in the proximal tubule of the kidney) (SLC4A5, rs7571842, rs10177833) have a higher risk for salt sensitivity and could benefit from a dietary reduction in salt. 6 Individuals who do not express these SNPs are considered normotensive salt resistant individuals. Since

### D. Conclusions

Inverse salt sensitivity represents a paradox in the field of salt sensitivity research. This emerging concept challenges the traditional understanding of sodium's impact on blood pressure regulation and how physicians may counsel their patients regarding the adjustment of sodium intake. Further studies are warranted to unravel the intricate mechanisms and clinical implications of inverse salt sensitivity, with the ultimate goal of improving the management and outcomes of individuals with hypertension. The fact that a patient can be normotensive and still be classified as SS or ISS and will have significant cardiovascular disease further emphasizes the need for novel diagnostic paradigms beyond the blood pressure cuff.

### Example 2

The devices, systems, apparatuses, modules, compositions, computer program products, non-transitory computer readable medium, and methods of various embodiments of the present disclosure may utilize aspects (such as devices, apparatuses, modules, systems, compositions, articles of manufacture, materials, computer program products, non-transitory computer readable medium, and methods) disclosed in the following references, applications, publications and patents and which are hereby incorporated by reference herein in their entireties (and which are not admitted to be prior art with respect to the present invention by inclusion in this section):
A. U.S. Utility patent application Ser. No. 15/651, 123, entitled “COMPOSITIONS AND METHODS FOR IDENTIFYING AND DIAGNOSING SALT SENSITIVITY OF BLOOD PRESSURE”, filed Jul. 17, 2017;
B. U.S. Utility patent application Ser. No. 14/395,596, entitled “COMPOSITIONS AND METHODS FOR IDENTIFYING AND DIAGNOSING SALT SENSITIVITY OF BLOOD PRESSURE”, filed Oct. 20, 2014; U.S. Pat. No. 9,708,664, issued Jul. 18, 2017; Publication No. 2015/0133414, May 14, 2015;
C. International Patent Application Serial No. PCT/US2013/037557, entitled “COMPOSITIONS AND METHODS FOR IDENTIFYING AND DIAGNOSING SALT SENSITIVITY OF BLOOD PRESSURE”, filed Apr. 22, 2013; Publication No. IVO 2013/159085, Oct. 24, 2013;
D. Felder R A, Gildea J J, Xu P, Yue W, Armando I, Carey R M, Jose P A. Inverse Salt Sensitivity of Blood Pressure: Mechanisms and Potential Relevance for Prevention of Cardiovascular Disease. Curr Hypertens Rep. 2022 September;24 (9): 361-374. doi: 10.1007/s11906-022-01201-9. Epub 2022 Jun. 16. PMID: 35708819; PMCID: PMC9728138;
E. Felder R A, Jose P A, Xu P, Gildea J J. The Renal Sodium Bicarbonate Cotransporter NBCe2: Is It a Major Contributor to Sodium and pH Homeostasis? Curr Hypertens Rep. 2016 September; 18 (9): 71. doi: 10.1007/s11906-016-0679-9. PMID: 27628629; PMCID: PMC6175536; and
F. Gildea J J, Xu P, Carlson J M, Gaglione R T, Bigler Wang D, Kemp B A, Reyes C M, McGrath H E, Carey R M, Jose P A, Felder R A. The sodium-bicarbonate cotransporter NBCe2 (slc4a5) expressed in human renal proximal tubules shows increased apical expression under high-salt conditions. Am J Physiol Regul Integr Comp Physiol. 2015 Dec. 1;309 (11): R1447-59. doi: 10.1152/ajpregu.00150.2015. Epub 2015 Oct. 7. PMID: 26447209; PMCID: PMC4698408.

### REFERENCES RELATED TO THE PRESENT DISCLOSURE

1. Minegishi S, Luft F C, Titze J, Kitada K. Sodium Handling and Interaction in Numerous Organs. Am J Hypertens. 2020;33:687-694. doi: 10.1093/ajh/hpaa049
2. Gildea J J, Lahiff D T, Van Sciver R E, Weiss R S, Shah N, McGrath H E, Schoeffel C D, Jose P A, Carey R M, Felder R A. A linear relationship between the ex-vivo sodium mediated expression of two sodium regulatory pathways as a surrogate marker of salt sensitivity of blood pressure in exfoliated human renal proximal tubule cells: the virtual renal biopsy. Clin Chim Acta. 2013;421:236-242. doi: 10.1016/j.cca.2013.02.021
3. Felder R A, White M J, Williams S M, Jose P A. Diagnostic tools for hypertension and salt sensitivity testing. Curr Opin Nephrol Hypertens. 2013;22:65-76. doi: 10.1097/MNH.0b013e32835b3693
4. Xu P, Sudarikova A V, Ilatovskaya D V, Gildea J J, Akhter M, Carey R M, Yue W, Jose P A, Felder R A. Epithelial Sodium Channel Alpha Subunit (αENaC) Is Associated with Inverse Salt Sensitivity of Blood Pressure. Biomedicines. 2022; 10. doi: 10.3390/biomedicines 10050981
5. Felder R A, Gildea J J, Xu P, Yue W, Armando I, Carey R M, Jose P A. Inverse Salt Sensitivity of Blood Pressure: Mechanisms and Potential Relevance for Prevention of Cardiovascular Disease. Curr Hypertens Rep. 2022;24:361-374. doi: 10.1007/s11906-022-01201-9
6. Carey R M, Schoeffel C D, Gildea J J, Jones J E, McGrath H E, Gordon L N, Park M J, Sobota R S, Underwood P C, Williams J, et al. Salt sensitivity of blood pressure is associated with polymorphisms in the sodium-bicarbonate cotransporter. Hypertension. 2012;60:1359-1366. doi: 10.1161/HYPERTENSIONAHA. 112.196071
7. Gildea J, Xu P, Schiermeyer K, Yue W, Carey R, Jose P, Felder R. The etiology of 7. inverse salt sensitivity of blood pressure: mirna-485-5p binds to the dopamine type 2 receptor (D2R) SNP rs6276 anddecreases D2R expression. Hypertension. 2020;76.
8. Graudal N, Jurgens G, Baslund B, Alderman M H. Compared with usual sodium intake, low-and excessive-sodium diets are associated with increased mortality: a meta-analysis. Am J Hypertens. 2014;27:1129-1137. doi: 10.1093/ajh/hpu028
9. Graudal N A, Hubeck-Graudal T, Jurgens G. Effects of low sodium diet versus high sodium diet on blood pressure, renin, aldosterone, catecholamines, cholesterol, and triglyceride. Cochrane Database Syst Rev. 2017;4: CD004022. doi: 10.1002/14651858.CD004022.pub4
10. Graudal N, Jürgens G. Conflicting Evidence on Health Effects Associated with Salt Reduction Calls for a Redesign of the Salt Dietary Guidelines. Prog Cardiovasc Dis. 2018;61:20-26. doi: 10.1016/j.pcad.2018.04.008
11. Gildea J J, Carlson J M, Schoeffel C D, Carey R M, Felder R A. Urinary exosome miRNome analysis and its applications to salt sensitivity of blood pressure. Clin Biochem. 2013;46:1131-1134. doi: 10.1016/j.clinbiochem.2013.05.052
12. M E M, J A D, M H R-G, CV V H, M R W, R V, A P, N I S, S S, N H B, et al. Determinants of blood pressure response to low-salt intake in a healthy adult population. Journal of Clinical Hypertension (Greenwich). 2011.
13. Cuka E, Simonini M, Lanzani C, Zagato L, Citterio L, Messaggio E, Faienza S, Brioni E, Hamlyn J M, Manunta P. Inverse salt sensitivity: an independent risk factor for cardiovascular damage in essential hypertension. J Hypertens. 2022;40:1504-1512. doi: 10.1097/HJH.0000000000003174
14. Juarez P D, Hood D B, Song M A, Ramesh A. Use of an Exposome Approach to Understand the Effects of Exposures From the Natural, Built, and Social Environments on Cardio-Vascular Disease Onset, Progression, and Outcomes. Front Public Health. 2020;8:379. doi: 10.3389/fpubh.2020.00379
15. Harrison D G, Coffman T M, Wilcox C S. Pathophysiology of Hypertension: The Mosaic Theory and Beyond. Circ Res. 2021; 128:847-863. doi: 10.1161/CIRCRESAHA. 121.318082
16. Ji W, Foo J N, O'Roak B J, Zhao H, Larson M G, Simon D B, Newton-Cheh C, State MW, Levy D, Lifton RP. Rare independent mutations in renal salt handling genes contribute to blood pressure variation. Nat Genet. 2008;40:592-599.
17. Harrison M, Maresso K, Broeckel U. Genetic determinants of hypertension: an update. Current Hypertension Reports. 2008; 10:488-495.
18. Kovesdy C P, Adebiyi A, Rosenbaum D, Jacobs J W, Quarles L D. Novel treatments from inhibition of the intestinal sodium-hydrogen exchanger 3. Int J Nephrol Renovasc Dis. 2021; 14:411-420. doi: 10.2147/IJNRD.S334024
19. Jose P A, Yang Z, Zeng C, Felder R A. The importance of the gastrorenal axis in the control of body sodium homeostasis. Exp Physiol. 2016; 101:465-470. doi: 10.1113/EP085286
20. Romberger N T, Stock J M, Patik J C, McMillan R K, Lennon S L, Edwards D G, Farquhar W B. Inverse salt sensitivity in normotensive adults: role of demographic factors. J Hypertens. 2023;41:934-940. doi: 10.1097/HJH.0000000000003413
21. Zheng Y, Horita S, Hara C, Kunimi M, Yamada H, Sugaya T, Goto A, Fujita T, Seki G. Biphasic regulation of renal proximal bicarbonate absorption by luminal AT (1A) receptor. J Am Soc Nephrol. 2003; 14:1116-1122. doi:

10.1097/01.asn.0000064700.58048.c1
22. Kotlo K, Shukla S, Tawar U, Skidgel R A, Danziger R S. Aminopeptidase N reduces basolateral Na+-K+-ATPase in proximal tubule cells. Am J Physiol Renal Physiol. 2007;293: F1047-1053. doi: 10.1152/ajprenal.00074.2007
23. Padia S H, Kemp B A, Howell N L, Fournie-Zaluski M C, Roques B P, Carey R M. Conversion of renal angiotensin II to angiotensin III is critical for AT2 receptor-mediated natriuresis in rats. Hypertension. 2008;51:460-465.
24. Yang S, Han Y, Zheng S, Kou X, Asico L D, Huang H, Gao Z, Jose P A, Zeng C. Enhanced Natriuresis and Diuresis in Wistar Rats Caused by the Costimulation of Renal Dopamine D3 and Angiotensin II Type 2 Receptors. Am J Hypertens. 2015;28:1267-1276. doi: 10.1093/ajh/hpv018
25. Overlack A, Ruppert M, Kolloch R, Göbel B, Kraft K, Diehl J, Schmitt W, Stumpe K O. Divergent hemodynamic and hormonal responses to varying salt intake in normotensive subjects. Hypertension. 1993;22:331-338.
26. Vaz de Castro P A S, Jose P A, Simões E Silva A C. Interactions between the intrarenal dopaminergic and the renin-angiotensin systems in the control of systemic arterial pressure. Clin Sci (Lond). 2022; 136:1205-1227. doi: 10.1042/CS20220338
27. Zeng C, Liu Y, Wang Z, He D, Huang L, Yu P, Zheng S, Jones J E, Asico L D, Hopfer U, et al. Activation of D3 dopamine receptor decreases angiotensin II type 1 receptor expression in rat renal proximal tubule cells. Circulation Research. 2006;99:494-500.
28 . Bek M J, Wang X, Asico L D, Jones J E, Zheng S, Li X, Eisner G M, Grandy D K, Carey R M, Soares-da-Silva P, et al. Angiotensin-II type 1 receptor-mediated hypertension in D4 dopamine receptor-deficient mice. Hypertension. 2006;47:288-295. doi: 10.1161/01.HYP.0000198427.96225.36
29. Gildea J J, Xu P, Schiermeyer K A, Yue W, Carey R M, Jose P A, Felder R A. Inverse Salt Sensitivity of Blood Pressure Is Associated with an Increased Renin-Angiotensin System Activity. Biomedicines. 2022; 10. doi: 10.3390/biomedicines10112811
30. Bitoun M, Levillain O, Tappaz M. Gene expression of the taurine transporter and taurine biosynthetic enzymes in rat kidney after antidiuresis and salt loading. Pflugers Arch. 2001;442:87-95. doi: 10.1007/s004240000506
31. Lahjouji K, Aouameur R, Bissonnette P, Coady M J, Bichet D G, Lapointe J Y. Expression and functionality of the Na+/myo-inositol cotransporter SMIT2 in rabbit kidney. Biochim Biophys Acta. 2007; 1768:1154-1159. doi: 10.1016/j.bbamem.2007.01.007
32. Bates M. MicroRNA Biomarkers: Pitfalls and Potential. IEEE Pulse. 2023; 14:16-19. doi: 10.1109/MPULS.2023.3294086
33. Khella H W, Bakhet M, Lichner Z, Romaschin A D, Jewett M A, Yousef G M. MicroRNAs in Kidney Disease: An Emerging Understanding. Am J Kidney Dis. 2012. doi: 10.1053/j.ajkd.2012.09.018
34. Yang J, Hall J E, Jose P A, Chen K, Zeng C. Comprehensive insights in GRK4 and hypertension: From mechanisms to potential therapeutics. Pharmacol Ther. 2022;239:108194. doi: 10.1016/j.pharmthera.2022.108194
35. Gildea J J, Kemp B A, Howell N L, Van Sciver R E, Carey R M, Felder R A. Inhibition of renal caveolin-1 reduces natriuresis and produces hypertension in sodium-loaded rats. Am J Physiol Renal Physiol. 2011;300: F914-920. doi: 10.1152/ajprenal.00380.2010
36. Gildea J J, Xu P, Kemp B A, Carey R M, Jose P A, Felder R A. The Dopamine D1 Receptor and Angiotensin II Type-2 Receptor are Required for Inhibition of Sodium Transport Through a Protein Phosphatase 2A Pathway. Hypertension. 2019;73:1258-1265. doi: 10.1161/HYPERTENSIONAHA. 119.12705
37. O'Donnell M, Mente A, Rangarajan S, McQueen M J, Wang X, Liu L, Yan H, Lee S F, Mony P, Devanath A, et al. Urinary sodium and potassium excretion, mortality, and cardiovascular events. N Engl J Med. 2014;371:612-623. doi: 10.1056/NEJMoa1311889
38. Mente A, O'Donnell M, Rangarajan S, McQueen M, Dagenais G, Wielgosz A, Lear S, Ah STL, Wei L, Diaz R, et al. Urinary sodium excretion, blood pressure, cardiovascular disease, and mortality: a community-level prospective epidemiological cohort study. Lancet. 2018;392:496-506. doi: 10.1016/S0140-6736 (18) 31376-X
39. O'Donnell M, Mente A, Rangarajan S, McQueen M J, O'Leary N, Yin L, Liu X, Swaminathan S, Khatib R, Rosengren A, et al. Joint association of urinary sodium and potassium excretion with cardiovascular events and mortality: prospective cohort study. BMJ. 2019;364:1772. doi: 10.1136/bmj.1772
40. Stolarz-Skrzypek K, Kuznetsova T, Thijs L, Tikhonoff V, Seidlerová J, Richart T, Jin Y, Olszanecka A, Malyutina S, Casiglia E, et al. Fatal and nonfatal outcomes, incidence of hypertension, and blood pressure changes in relation to urinary sodium excretion. JAMA. 2011;305:1777-1785. doi: 10.1001/jama.2011.574
41. Weinberger M H, Fineberg N S, Fineberg S E, Weinberger M. Salt sensitivity, pulse pressure, and death in normal and hypertensive humans. Hypertension. 2001;37:429-432.
42. Weinberger M H. Salt sensitivity is associated with an increased mortality in both normal and hypertensive humans. J Clin Hypertens (Greenwich). 2002;4:274-276.
43. Gildea J J, Xu P, Carlson J M, Gaglione R T, Bigler Wang D, Kemp B A, Reyes C M, McGrath H E, Carey R M, Jose P A, et al. The sodium-bicarbonate cotransporter NBCe2 (slc4a5) expressed in human renal proximal tubules shows increased apical expression under high-salt conditions. Am J Physiol Regul Integr Comp Physiol. 2015;309: R1447-1459. doi: 10.1152/ajpregu.00150.2015
44. Felder R, Huntley D, Surace M, Jose P, Carey R, Gildea J. Culture of exfoliated human renal proximal tubular cells in urine from normal subjects enrolled in a genetic study of salt sensitivity. Hypertension. 2006;48: e64.
45. Ishigami J, Charleston J, Miller E R, Matsushita K, Appel L J, Brady T M. Effects of Cuff Size on the Accuracy of Blood Pressure Readings: The Cuff (SZ) Randomized Crossover Trial. JAMA Intern Med. 2023. doi: 10.1001/jamainternmed.2023.3264
46. Mitchell B D, McArdle P F, Shen H, Rampersaud E, Pollin T I, Bielak L F, Jaquish C, Douglas J A, Roy-Gagnon M H, Sack P, et al. The genetic response to short-term interventions affecting cardiovascular function: rationale and design of the Heredity and Phenotype Intervention (HAPI) Heart Study. Am Heart J. 2008; 155:823-828. doi: 10.1016/j.ahj.2008.01.019
47. Sun B, Williams J S, Pojoga L, Chamarthi B, Lasky-Su J, Raby B A, Hopkins P N, Jeunemaitre X, Brown N J, Ferri C, et al. Renin gene polymorphism: its relationship to hypertension, renin levels and vascular responses. J Renin Angiotensin Aldosterone Syst. 2011;12:564-571. doi: 10.1177/1470320311405873
48. Felder R A, Sanada H, Xu J, Yu P Y, Wang Z, Watanabe H, Asico L D, Wang W, Zheng S, Yamaguchi I, et al. G protein-coupled receptor kinase 4 gene variants in human essential hypertension. Proc Natl Acad Sci USA. 2002;99:3872-3877.
49. Sanada H, Yatabe J, Midorikawa S, Hashimoto S, Watanabe T, Moore J H, Ritchie M D, Williams S M, Pezzullo J C, Sasaki M, et al. Single-nucleotide polymorphisms for diagnosis of salt-sensitive hypertension. Clin Chem. 2006;52:352-360.
50. Felder R A, Jose P A, Xu P, Gildea J J. The Renal Sodium Bicarbonate Cotransporter NBCe2: Is It a Major Contributor to Sodium and pH Homeostasis? Curr Hypertens Rep. 2016; 18:71. doi: 10.1007/s11906-016-0679-9
51. Rayner B, Ramesar R, Steyn K, Levitt N, Lombard C, Charlton K. G-protein-coupled receptor kinase 4 polymorphisms predict blood pressure response to dietary modification in Black patients with mild-to-moderate hypertension. J Hum Hypertens. 2012;26:334-339. doi: 10.1038/jhh.2011.33
52. Bhatnagar V, O'Connor D T, Brophy V H, Schork N J, Richard E, Salem R M, Nievergelt C M, Bakris G L, Middleton J P, Norris K C, et al. G-protein-coupled receptor kinase 4 polymorphisms and blood pressure response to metoprolol among African Americans: sex-specificity and interactions. Am J Hypertens. 2009;22:332-338. doi: 10.1038/ajh.2008.341
53. Vandell A G, Lobmeyer M T, Gawronski B E, Langaee T Y, Gong Y, Gums J G, Beitelshees A L, Turner S T, Chapman A B, Cooper-DeHoff R M, et al. G protein receptor kinase 4 polymorphisms: 3-blocker pharmacogenetics and treatment-related outcomes in hypertension. Hypertension. 2012;60:957-964. doi: 10.1161/HYPERTENSIONAHA. 112.198721
54. Wagner F, Malice M P, Wiegert E, McGrath H E, Gildea J, Mitta S, Van Dyck K, De Lepeleire I, Johnson-Levonas A O, Sisk C M, et al. A comparison of the natriuretic and kaliuretic effects of cicletanine and hydrochlorothiazide in prehypertensive and hypertensive humans. J Hypertens. 2012;30:819-827. doi: 10.1097/HJH.0b013e32835022a8

It should be emphasized that the above-described embodiments are merely examples of possible implementations. Many variations and modifications may be made to the above-described embodiments without departing from the principles of the present disclosure. All such modifications and variations are intended to be included herein within the scope of this disclosure and protected by the following claims.

At least the following is claimed:

1. A method of determining the salt index of a subject, comprising:
classifying a subject's blood pressure status as normotensive or hypertensive according to one or more first blood pressure measurements of the subject, measuring the rate of sodium excretion from the body of the subject over a period of time following a high and then low sodium diets in random order, wherein excessive rapid sodium excretion from the body of the subject determines an inverse salt-sensitive status (ISS) for the subject, and a slow excretion of sodium determines salt-sensitive status relative to the rate of sodium excretion in one or more salt resistant (SR) individuals, or both;
determining the future hypertension, salt sensitivity (SS), or inverse salt-sensitivity (ISS) status of the subject by identifying, in a biological sample from the subject, the presence of:
one or more sodium-bicarbonate cotransporter NBCe2 (SLC4a5) polymorphisms for SS status determination,
one or more G protein-coupled receptor kinase 4 (GRK4) polymorphisms for hypertensive blood pressure status determination and SS status determination,
one or more sodium channel epithelial 1 subunit alpha (αENaC) polymorphisms for ISS status determination, and
one or more dopamine receptor D2 (DRD2) polymorphisms for ISS status determination.
prescribing a low salt diet or high salt diet to the subject based on the subject's blood pressure status and determined future hypertension, salt sensitivity, or inverse salt-sensitivity status of the subject, or pharmacogenetic testing, wherein:
a high salt diet is prescribed to the normotensive subject having one or more SLC4a5 polymorphisms,
a low salt diet is prescribed to the hypertensive subject having three or more GRK4 polymorphisms,
a low salt diet is prescribed to the hypertensive subject having one or more αENaC polymorphisms and one or more GRK4 polymorphisms, and
a high salt diet is prescribed to the hypertensive subject having one or more DRD2 polymorphisms;
classifying a subject's salt-sensitive blood pressure status as salt-sensitive, salt-resistance, or inverse-salt sensitive after following the prescribed low-salt or high-salt diet according to one or more second blood pressure measurements of the subject;
identifying, from the one or more second blood pressure measurements of the subject, a salt-sensitivity status of the subject after following the prescribed low-salt or high-salt diet as being:
salt-sensitive if mean arterial pressure (MAP) is controlled by a dietary salt reduction;
salt-resistant if MAP unaffected by sodium;
salt-resistant controlled by diet;
salt-resistant essential hypertensive;
inverse salt-sensitive controlled by diet; or
salt-resistant with essential hypertension of the hypertensive subject not having a MAP not controlled by diet.

2. The method of claim 1, wherein the subject is hypertensive if the one or more first blood pressure measurements are ≥130/80 mm Hg systolic/diastolic or is normotensive if the one or more first blood pressure measurements are <130/80 mm Hg systolic/diastolic.

3. The method of claim 1, wherein the sample is a urine sample or spot urine sample.

4. The method of claim 1, wherein the high salt diet comprises high salt intake of about 300 mmol/day and the low salt diet comprises low salt intake of about 10 mm/day.

5. The method of claim 1, wherein the subject is on the high salt diet or low salt diet for at least 7 days.

6. The method of claim 1, wherein the subject is salt-sensitive if the one or more first second pressure measurements demonstrate a ≥+5 mmHg change in MAP after the salt-diet, inverse salt-sensitive if the one or more first second pressure measurements demonstrate a ≥−5 mmHg change in MAP after the salt-diet, and salt-resistant if the subject exhibits <+5 mmHg change in MAP or <−5 mmHg change in MAP after the salt diet.

7. The method of claim 1, further comprising:
determining, from a sample of a normotensive subject not having one or more SLC4A5 polymorphisms:
a level of dopamine type 1 receptor (D₁R) expression, D₁R activity, or both,
a level of Ang II type 2 receptor (AT₂R) expression, AT₂R activity, or both,
determining the presence of one or more micro ribonucleic acids (miRNA) associated with salt-sensitivity or inverse salt-sensitivity, or
any combination of any thereof;
identifying the subject as salt-sensitive if the subject has:
altered D₁R expression, activity, or both compared to a wild-type normotensive control,
altered AT₂R expression, activity, or both compared to a wild-type normotensive control; or
the presence of one or more micro ribonucleic acid (miRNA) associated with salt-sensitivity or inverse salt-sensitivity.

8. The method of claim 7, wherein one or more miRNA associated with salt-sensitivity or inverse salt-sensitivity comprises at least miR-485-5p.

9. The method of claim 8, further comprising, prescribing a dietary salt reduction to the normotensive subject identified as salt-sensitive.

10. The method of claim 1, further comprising:
prescribing, administering, or both:
an anti-hypertensive pharmacogenetic test for anti-hypertensive therapeutics for the subject identified as salt-resistant with essential hypertension.

11. The method of claim 1, further comprising:
prescribing, to the subject with a classified salt sensitivity or salt-resistant with essential hypertension, at least:
a low salt diet, β-adrenergic blockers, or both, to the subject having wild-type GRK4 and the GRK4 polymorphisms R65L, A142V, and A486V;
a non-β-adrenergic blocker to the subject having one or more wild-type GRK4 allelic variants and the GRK4 polymorphisms R65L and A142V;
one or more β-adrenergic blockers, one or more AT₁R blockers, or any combination of any thereof to the subject having one or more wild-type GRK4 allelic variants and the GRK4 polymorphism A142V;
a low-salt diet and diuretics to the subject having one or more wild-type GRK4 allelic variants and at least the GRK4 polymorphisms R65L, A142V, A486V; and
one or more AT₁R blockers and one or more diuretics to the subject having one or more wild-type GRK4 allelic variants and at least the GRK4 polymorphisms A142V and A486V.

12. The method of claim 1, further comprising:
determining, in the biological sample of the subject or a second biological sample from the subject:
the presence of one or more polymorphisms in solute carrier family 5 member 3 (SLC5A3), the presence of one or more polymorphisms in solute carrier family 5 member 11 (SLC5A11), or both.

13. The method of claim 12, wherein at least one of the one or more SLC5A11 polymorphisms comprise rs11074656.

14. A method of determining the salt index of a subject, comprising:
classifying a subject's blood pressure status as normotensive or hypertensive according to one or more first blood pressure measurements of the subject;
determining the future hypertension, salt sensitivity, or inverse salt-sensitivity status of the subject by identifying, in a biological sample from the subject: the presence of:
one or more SLC4a5 polymorphisms,
one or more GRK4 polymorphisms,
one or more αENaC polymorphisms, and
one or more DRD2 polymorphisms;
prescribing a low salt diet or high salt diet to the subject based on the subject's blood pressure status and determined future hypertension, salt sensitivity, or inverse salt-sensitivity status of the subject, or pharmacogenetic testing, wherein:
a high salt diet is prescribed to the normotensive subject having one or more SLC4a5 polymorphisms,
a low salt diet is prescribed to the hypertensive subject having three or more GRK4 polymorphisms,
a low salt diet is prescribed to the hypertensive subject having one or more αENaC polymorphisms and one or more GRK4 polymorphisms,
a high salt diet is prescribed to the hypertensive subject having one or more DRD2 polymorphisms;
classifying a subject's salt-sensitive blood pressure status as salt-sensitive, salt-resistance, or inverse-salt sensitive after following the prescribed salt diet according to one or more second blood pressure measurements of the subject, wherein the subject is:
salt-sensitive if the one or more first second pressure measurements demonstrate a ≥+5 mmHg change in MAP after the salt-diet,
inverse salt-sensitive if the one or more first second pressure measurements demonstrate a ≥−5 mmHg change in MAP after the salt-diet, and
salt-resistant if the subject exhibits <+5 mmHg change in MAP or <−5 mmHg change in MAP after the salt diet;
identifying, from the one or more second blood pressure measurements of the subject, a salt-sensitivity status of the subject after following the prescribed salt diet as being:
salt-sensitive if mean arterial pressure (MAP) is controlled by a dietary salt reduction,
salt-resistant if MAP unaffected by sodium,
salt-resistant controlled by diet,
salt-resistant essential hypertensive,
inverse salt-sensitive controlled by diet; or
salt-resistant with essential hypertension to the hypertensive subject not having a MAP not controlled by diet; and
prescribing, administering, or both, an anti-hypertensive pharmacogenetic test for
anti-hypertensive therapeutics if the subject is identified as salt-resistant with essential hypertension, wherein the anti-hypertensive pharmacogenetic test comprises determining the presence of one or more GRK4 polymorphisms from the identified one or more GRK4 polymorphisms of the biological sample or from a second biological sample from the subject; and
prescribing, to the subject with a classified salt sensitivity of salt-resistant with essential hypertension, at least:
a low salt diet, β-adrenergic blockers, or both, to the subject having wild-type GRK4 and the GRK4 polymorphisms R65L, A142V, and A486V;
a non-β-adrenergic blocker to the subject having one or more wild-type GRK4 allelic variants and the GRK4 polymorphisms R65L and A142V;
one or more β-adrenergic blockers, one or more AT₁R blockers, or any combination of any thereof to the subject having one or more wild-type GRK4 allelic variants and the GRK4 polymorphism A142V;
a low-salt diet and diuretics to the subject having one or more wild-type GRK4 allelic variants and at least the GRK4 polymorphisms R65L, A142V, A486V; and
one or more AT₁R blockers and one or more diuretics to the subject having one or more wild-type GRK4 allelic variants and at least the GRK4 polymorphisms A142V and A486V.

15. The method of claim 14, further comprising:
determining, in the biological sample of the subject or a second biological sample from the subject:
the presence of one or more polymorphisms in SLC5A3, the presence of one or more polymorphisms in SLC5A11, or both.

16. A kit, comprising:
one or more primers or probes for determining, in a biological sample from a subject, the presence of:
one or more SLC4a5 polymorphisms,
one or more GRK4 polymorphisms,
one or more αENaC polymorphisms,
one or more DRD2 polymorphisms,
or any combination of any thereof; and
instructions for use, wherein the instructions for use comprise instructions for the user of the kit to perform at least the method of claim 1.

17. The kit of claim 16, further comprising a sample collection device.

18. The kit of claim 16, wherein the instructions for use further comprise instructions for using the sample collection device.

19. The kit of claim 16, further comprising one or more primers or probes for determining, in a biological sample from a subject, the presence of one or more polymorphisms in SLC5A3, the presence of one or more polymorphisms in SLC5A11, or both.

20. The kit of claim 16, further comprising:
one or more primers or probes for determining the level of D₁R expression,
one or more primers or probes for determining the level of AT₂R expression,
one or more primers or probes for determining the presence of one or more micro ribonucleic acid (miRNA) associated with salt-sensitivity or inverse salt-sensitivity, or
any combination of any thereof.
